(19) United States
(12) Patent Application Publication
(10) Pub. No.: US 2022/0110789 A1
(43) Pub. Date: Apr. 14, 2022

(54) Methods, Computer-Readable Media, and Systems for Treating a Cornea

(71) Applicant: The Trustees of Columbia University in the City of New York, New York, NY (US)

(72) Inventor: Sinisa VUKELIC, New York, NY (US)

(73) Assignee: The Trustees of Columbia University in the City of New York, New York, NY (US)

(21) Appl. No.: 17/298,196

(22) PCT Filed: Nov. 26, 2019
(86) PCT No.: PCT/US2019/063320

§ 371 Date: May 28, 2021

Related U.S. Application Data
(60) Provisional application No. 62/773,000, filed on Nov. 29, 2018.

(51) Int. Cl. A61F 9/009

(52) U.S. Cl. CPC A61F 9/009; A61F 2009/00882

(57) ABSTRACT

Femtosecond laser may be used to crosslink corneal collagen in absence of photosensitizers to correct refractive errors and enhance corneal mechanical properties of tissues, such as the cornea. The treatment time is reduced by defining treatment layers in the tissue being treated and focusing the laser at selected layers to effect treatment at the multiple layers. Volumetric exposure to the laser has been executed by treating multiple planar areas at varying depths, measured from the surface of the treated tissue.

### CROSS-REFERENCE TO RELATED APPLICATION

This application claims the benefit of priority to U.S. Provisional Patent Application Ser. No. 62/773,000, filed Nov. 29, 2018. The entire content of this application is hereby incorporated by reference herein.

### BACKGROUND OF THE INVENTION

Collagen is an abundant protein in animals. The mechanical properties and structural stability of collagen based tissues, such as corneal tissue, can be influenced by increasing collagen cross-links (CXL), in the form of intra- or inter-molecule chemical bonds.

### SUMMARY OF THE INVENTION

An aspect of the invention provides a method of altering curvature of a cornea. The method includes receiving one or more measurements of topography of a cornea, calculating a pattern defining locations and amounts of cross-linking required to achieve a desired level of vision correction, wherein the amounts of cross-linking are, at least in part, a function of a number of overlapping treatment layers having different z depths at a given coordinate, and controlling a light source to apply light energy pulses to the cornea to cross-link collagen in accordance with the pattern.

This aspect of the invention can have a variety of embodiments. In some embodiments, the method may include receiving one or more measurements of thickness of the cornea, wherein the amounts of cross-linking are also, at least in part, a function of the thickness of the cornea.

In some embodiments, multiple treatment layers are defined based on their respective z depths measured relative to the surface of the cornea, and a subset of the defined treatment layers is treated to achieve a desired effect in the cornea. In some embodiments, the subset of the defined treatment layers includes layers that are adjacent to each other in the z direction. In some embodiments, the subset of the defined layers includes layers that are not adjacent to each other but are instead spaced apart to achieve a desired effect in the cornea.

In some embodiments, the light energy pulses are applied in the absence of an exogenous photosensitizer.

In some embodiments, the light energy pulses ionize water molecules within the cornea to generate reactive oxygen species.

In some embodiments, the light energy pulses have a wavelength that is not absorbed by amino acids in collagen.

In some embodiments, the light energy pulses have a wavelength that is absorbed by amino acids in collagen.

In some embodiments, the method further includes applying an exogenous photosensitizer to the cornea before controlling the light source.

In some embodiments, the exogenous photosensitizer is riboflavin.

In some embodiments, the light source is a laser.

In some embodiments, the laser is a femtosecond laser.

In some embodiments, the light energy pulses have an average power output between about 10 mW and about 100 mW.

In some embodiments, the light energy pulses have a pulse energy between about 0.1 nJ and about 10 nJ.

In some embodiments, the light energy pulses have a wavelength between about 600 nm and about 1600 nm.

Another aspect of the invention provides a system for treating a cornea. The system includes a light source configured to project light energy pulses onto at least a portion of a cornea and a controller programmed to calculate the pattern and control the light source in accordance with any of embodiments above.

Another aspect of the invention provides a system for adapting a laser system for treating a cornea. The system includes laser modification optics adapted and configured to adjust laser output of the laser system and a controller programmed to calculate the pattern and control the laser modification optics as the light source in accordance with any of the embodiments above.

Another aspect of the invention provides a method of treating a cornea. The method includes controlling a light source to apply light energy pulses to a single corneal layer selected from the group consisting of: an anterior corneal layer and a posterior corneal layer. The light energy pulses: are below an optical breakdown threshold for the cornea; and ionize water molecules within the treated corneal layer to generate reactive oxygen species that cross-link collagen within the single corneal layer.

This aspect of the invention can have a variety of embodiments. The anterior corneal layer can extend between an anterior surface of the cornea and about 200 microns from the anterior surface. The posterior corneal layer can extend between a posterior surface of the cornea and about 200 microns from the posterior surface.

Another aspect of the invention provides a method of treating a cornea. The method includes controlling a light source to apply light energy pulses to at least a corneal stroma layer of a cornea. The light energy pulses: are below an optical breakdown threshold for the cornea; and ionize water molecules within the treated corneal stromal layer to generate reactive oxygen species that cross-link collagen within the cornea.

These aspects can have a variety of embodiments. The light source can be a laser. The laser can be a femtosecond laser.

The light energy pulses can have an average power output between about 10 mW and about 100 mW. The light energy pulses can have a pulse energy between about 0.1 nJ and about 10 nJ. The light energy pulses can have a wavelength between about 600 nm and about 1600 nm. The light energy pulses can have a wavelength that is not absorbed by amino acids in collagen.

The light energy pulses can be applied in a pattern. The pattern can extend across a center of an iris posterior to the cornea. The pattern can surround, but not extend across a center of an iris posterior to the cornea.

The method can treat keratoconus or alter curvature of the cornea.

Another aspect of the invention provides a system for treating a cornea. The system includes: a light source configured to project light energy pulses onto at least a portion of a cornea; and a controller programmed to control the light source in accordance with any of the methods described herein.

Another aspect of the invention provides a system for adapting a laser system for treating a cornea. The system includes: laser modification optics adapted and configured to adjust laser output of the laser system; and a controller programmed to control the laser modification optics as the light source in accordance with any of the methods described herein.

Another aspect of the invention provides a method of treating a cornea where the method includes flattening the cornea with a material that transmits light, generating pulses with a tunable femtosecond laser system, focusing the generated pulses on a focal volme at a specific depth within the cornea as measured from a surface of the eye, moving the focal volume at the specific depth to define a treatment pattern, and repeating the focusing and moving steps at multiple different depths. This aspect can have multiple embodiments described below. It is understood that each embodiment below can be combined with all of the other embodiments of this aspect.

In some embodiments, the focusing is achieved by using an aspheric lens.

In some embodiments, the moving of the focus point takes place at 30 mm/s in a direction parallel with the material used to flatten the cornea.

In some embodiments, adjacent ones of the multiple different depths are separated by 50 μm.

In some embodiments, the flattening the cornea includes pressing a glass coverslip against the cornea.

In some embodiments, the generating pulses is performed with a temporal pulse width of 140 fs at 80 MHz repetition rate with central wavelength set to 1060 nm.

In some embodiments, the treatment pattern is a zig-zag pattern.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1 illustrates a flow diagram of a cross-linking process applied to the cornea according to an embodiment of the invention.

FIGS. 2A and 2B are schematics illustrating a mechanism of action according to an embodiment of the invention.

FIGS. 3A-3D depict systems (FIG. 3A), topography controls (FIG. 3B), and multiple beam architectures (FIGS. 3C and 3D) for treating a cornea according to embodiments of the invention.

FIGS. 4A-4C depict the time course of the change in normalized effective refractive power (EFR) after the laser treatment of porcine eyes ex vivo. FIG. 4A depicts a flattening treatment (e.g., for myopia). FIG. 4B depicts a steepening treatment (e.g., for hyperopia). FIG. 4C depicts a control study analyzing the effects of the treatment protocol. The treatment involves applying laser pulses such that the path of the laser follows a zigzag trajectory, thus treating a planar area at a specific depth. The treatment is repeated at different depths, effectively inducing multiple “treatment layers”. Multiple treatment layers parallel to the superficial surface were created, with a distance of 50 μm between consecutive planes. The y axis corresponds to effective refractive power normalized against diopter (D) values before treatment. Changes in the refractive power of the eye relative to the measurement performed immediately before treatment are shown. The error bars indicate the standard deviation.

FIGS. 5A and 5B depict corneal topography of isolated porcine eyes (FIG. 5A) before and (FIG. 5B) after laser treatment.

FIGS. 5C and 5D depict results shown paired with virtual vision in FIG. 5C and FIG. 5D to demonstrate the effects of the refractive error correction applied. The corneal elevation maps show effective refractive powers of 45 diopters before and 43.5 diopters after treatment. The virtual vision for the corneal effective refractive powers shown corresponds to 45 diopters in FIG. 5C and 43.5 diopters FIG. 5D, assuming that 43.5 diopters corresponds to a visual acuity of 20/20 (normal vision).

FIG. 6 depicts isolated rabbit eyes in 3D-printed holders connected with an IV pressure control system.

FIG. 7A depicts an experimental set-up. FIG. 7B depicts a treatment system according to an embodiment of the invention.

FIG. 8 depicts a laser treatment pattern according to an embodiment of the invention. Five mutually independent layers were treated with 50 μm in between two layers and each layer was treated through a zigzag path.

FIGS. 9A-9C depict time-histories of the change in normalized effective refractive power (EFR) after the laser treatment of porcine eyes ex vivo for treatment from anterior surface (FIG. 9A), treatment from posterior surface (FIG. 9B), and control treatment (FIG. 9C). Treatment consists of applying laser pulses such that the laser path follows a zigzag pattern, thus treating a planar surface at the specific depth. The treatment is repeated at different depths, effectively inducing “treatment layers”. Multiple treatment layers parallel to the superficial surface were applied with 50 μm distance between two consecutive planes.

FIGS. 10A-C illustrate two-photon fluorescence (TPF) images of (a) untreated control, (b) anterior laser treated, and (c) posterior laser treated cross sections of ex vivo rabbit eyes. The central zone of the untreated control or laser irradiated corneal tissues were imaged. The control sample and the untreated region of the laser irradiated specimen show approximately the same properties. Three different intensity lines were drawn through the whole corneal thickness (location indicated as three arrows in FIGS. 10A-C).

FIG. 11 is a chart of the average gray value for intensity lines of the three groups in FIG. 10. The chart clearly illustrates the treatment-induced intensity change. The anterior treated group showed an increased intensity from superficial surface to a depth around 200 and the posterior treated group showed a similar trend from the bottom surface to a depth around 200 μm in the cornea, whereas the untreated control group presented a relatively stable signal intensity throughout the whole corneal thickness. Boxed regions in FIGS. 10A-C indicated the histogram acquirements for each group.

FIG. 12 depicts the average pixel value for all the three groups from FIG. 10.

FIGS. 13A-13C depict histological sections of H&E -stained samples of untreated control (FIG. 13A), anterior treated (FIG. 13B), and posterior treated (FIG. 13C) rabbit corneas. The scale bar is 100 μm.

FIGS. 14A-14F provide representative CLSM (Confocal Laser Scanning Microscopy) images of the ex vivo untreated control (FIGS. 14A and 14D), anterior laser treated (FIGS. 14B and 14E) and posterior treated (FIGS. 14C and 14F) rabbit eyes. The scale bar is 100 μm.

FIG. 15 is chart of average normalized refractive power changes for 24 hours with 4 hours between each time point, for 5 control eyes and 4 eyes for each treatment group (a total of 5+4*5=25 eyes). Error bars are shown as standard deviation.

FIG. 16 depicts average diopter changes for each treatment group. Error bars are shown as standard deviation.

FIGS. 17A-F illustrate results of a controlled parametric study on porcine eyes with 0, 1, and 2 treatment layers.

FIGS. 18A-F illustrate results of a controlled parametric study on porcine eyes with 3, 4, and 5 treatment layers.

FIGS. 19A-H and 19J-M illustrate results of tests of mechanical properties of corneas.

FIGS. 20A-H and 20J-M illustrate additional results of tests of mechanical properties of corneas.

FIGS. 21A-F illustrate representative pressure displacement curves.

FIGS. 22A-F illustrate displacement position maps of corneas subject to inflation tests.

FIGS. 23A-F illustrate Youngs' Modulus maps of corneas subject to inflation tests.

### DEFINITIONS

The instant invention is most clearly understood with reference to the following definitions. As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION

Embodiments of the invention provide methods, computer-readable media, and systems for treating a cornea by applying light to one or more corneal layer(s) to cross-link collagen. The cross-linking is performed to achieve a desired level of vision correction and/or the desired level of stiffening of the cornea. The amount of cross-linking is, at least in part, a function of a number of overlapping treatment layers having different z depths at a given coordinate. A treatment layer is a selected depth, measured from the posterior or anterior surface of the cornea, at which treatment light (such as a laser) is focused. It will be understood that the aperture used to focus the treatment light allows definition of such layers within the cornea.

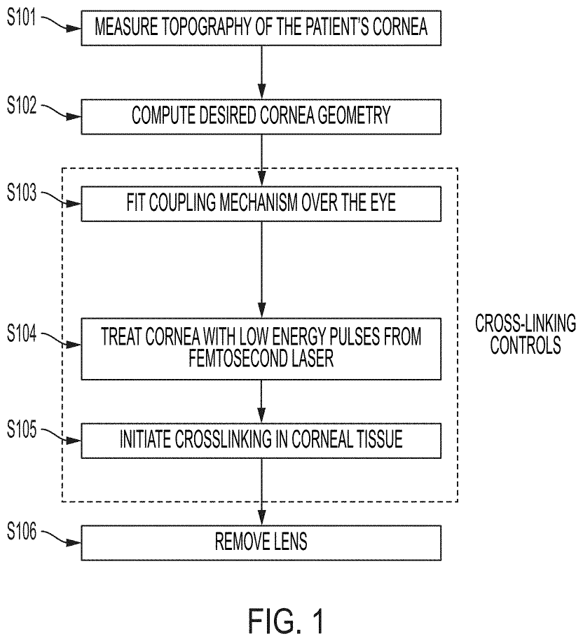

Referring now to FIG. 1, an example of a method of inducing cross-linking in corneal tissue is shown. In step S101, the topography of the patient's cornea can be measured. In step S102, the desired cornea geometry can be computed. In some embodiments, the goal may to strengthen the cornea without changing its shape. In this case, step S102 can compute the desired locations to strengthen without changing the corneal shape. In an embodiment, a coupling mechanism can be placed over the eye to be treated in step S103. However, this method is not limited to such embodiments, and the cornea can be treated without a coupling mechanism. In step S104, a light source can be driven to emit low energy pulses that are guided and focused as discussed herein. As shown in step S105, the interaction of the pulse laser with the aqueous medium in and around the tissue initiates cross-linking. In step S106, a lens of the coupling mechanism (if one was used) is removed from the cornea.

Exemplary Therapies

The methods and systems can be used to treat various corneal disorders including keratoconus, myopia, hyperopia, stigmatism, irregular astigmatism, and other ectatic diseases (e.g., those that result from a weakened corneal stroma). The methods and systems can also be used in refractive surgery, e.g., to modify corneal curvature or correct irregular surfaces and higher-order optical aberrations.

Exemplary Irradiation Parameters

As described in International Publication No. WO 2017/070637 and U.S. Patent Application Publication Nos. 2018/0193188 and 2018/0221201, corneal cross-linking can be achieved without the need for exogenous photosensitizers such as riboflavin by ionizing water within corneal tissue to generate reactive oxygen species that cross-link collagen strands. Cross-linking can be achieved over a broad range of wavelengths including those that are not absorbed by amino acids within collagen strands. For example, the laser wavelength can be in the range from about 250 nm to about 1600 nm. In some embodiments, the laser wavelength can be in the range from about 250 nm to about 1600 nm, but excluding wavelengths between 260-290 nm, 520-580 nm, 780-870 nm, and 1040-1160 nm.

By controlling pulse energy to be below the optical breakdown threshold of collagen (about 1.0×1013 W cm-2), the mechanical properties of the collagen can be modified without modifying the refractive index of the collagen. For example, the curvature of the cornea can be modified to change the refractive power of the cornea.

Ionization can be created within tissue using a laser emission that is absorbed by the tissue. For example, the laser emission can be based on ultrashort laser pulses. As used herein, the phrase “ultrashort laser pulses” includes emissions in the femtosecond, picosecond, and nanosecond ranges. Nonlinear absorption of laser emissions can occur, in part, due to the highly compressed nature of the light pulses, allowing treatments of the interior of a transparent dielectric, such as corneal tissue, without affecting the surface layer. In some embodiments, a tunable femtosecond laser system (e.g., Coherent, Chameleon Ultra II, Santa Clara, Calif.) may be employed to generate laser pulses with temporal pulse width of 140 fs (femtoseconds) at 80 MHz repetition rate, and with central wavelength set to 1060 nm. The laser may be coupled through a single mode fiber cable (e.g., P1-980A-FC-1 single mode fiber patch cable, Thorlabs, Newton, N.J.) through an optical setup as described below.

The ultrashort laser pulse can induce low-density plasma that ionizes water molecules within the tissue, while still operating below the energy level required for optical breakdown. Optical breakdown is the effect of an ultrafast laser focused in the interior of collagen-rich tissue, where photoionization triggers non-linear absorption. Continued supply of incoming photons leads to the buildup of free electrons, further leading to avalanche ionization, which enhances the growth of free electron density resulting in formation of plasma. As contrasted from the low-density plasma, high-density, opaque plasma strongly absorbs laser energy through free carrier absorption. The high-density plasma expands rapidly, creating a shockwave that propagates into surrounding material, creating optical breakdown.

Collagen cross-linking can be safely induced when the laser is operated below optical breakdown level in the so-called “low-density plasma” regime. For example, the laser emission, as defined by its wavelength, temporal pulse width, and pulse energy, as well as the numerical aperture of the scanning objective and the scanning speed should be high enough to induce ionization of water molecules in the collagen rich tissue, but below optical breakdown level. Further, such ionization can be induced in the cornea without reducing the transparency of the cornea.

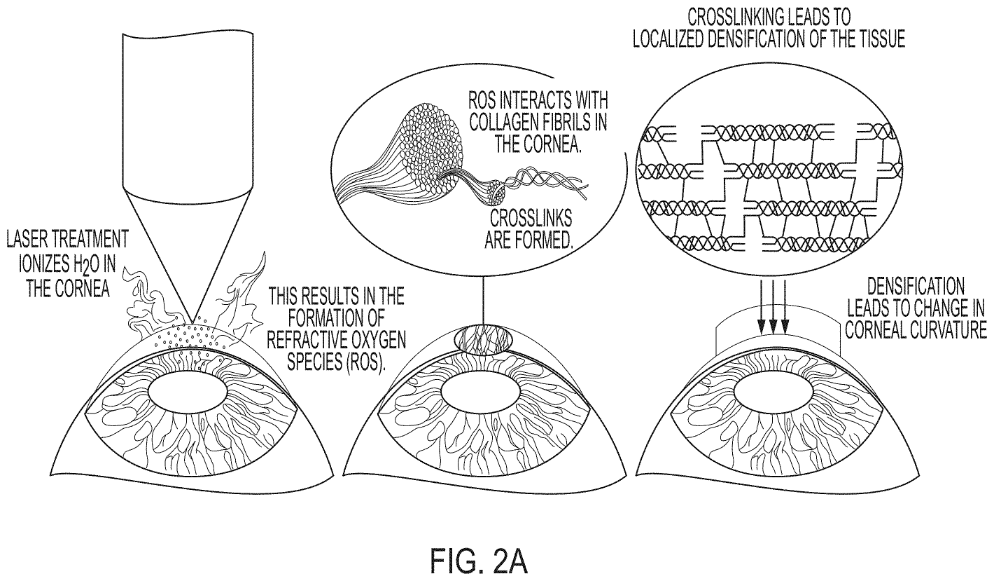

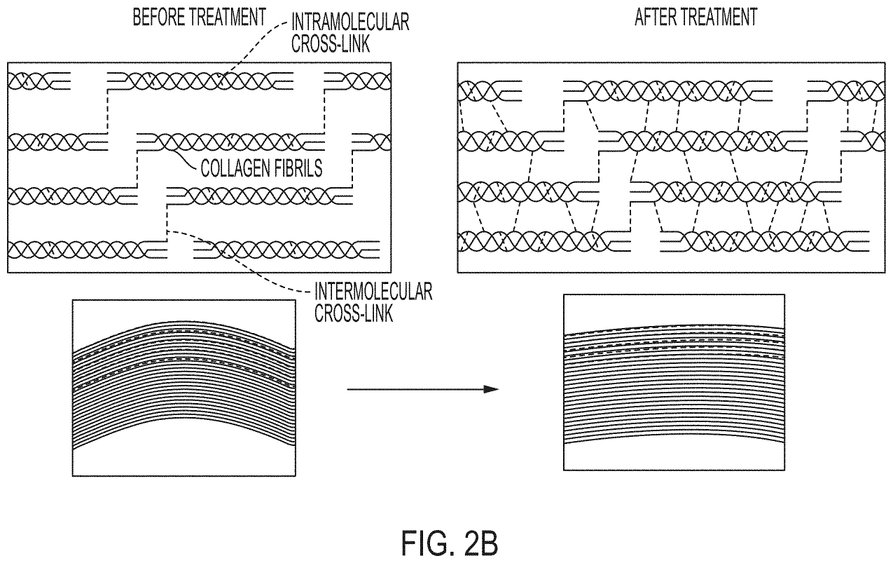

Without being bound by theory, the ionization can cause the formation of reactive oxygen products, such as singlet oxygen, OH-, and H202, which, in turn, can interact with collagen and increase cross-linking in the fibrils, as shown in FIGS. 2A and 2B. Additionally, singlet oxygen generated by the ionization can inactivate collagenase and have a germicidal effect, increasing the utility of these methods for clinical applications. In embodiments, deuterium oxide can be introduced onto the cornea to prolong half-life of the produced singlet oxygen, thereby increasing cross-linking efficiency.

In certain aspects, the presently disclosed subject matter provides methods of inducing such ionization. The methods can be used in the treatment of various ectatic diseases or during refractive surgery. The methods can include modifying the corneal curvature by inducing selective corneal cross-linking.

Exemplary Corneal Layers

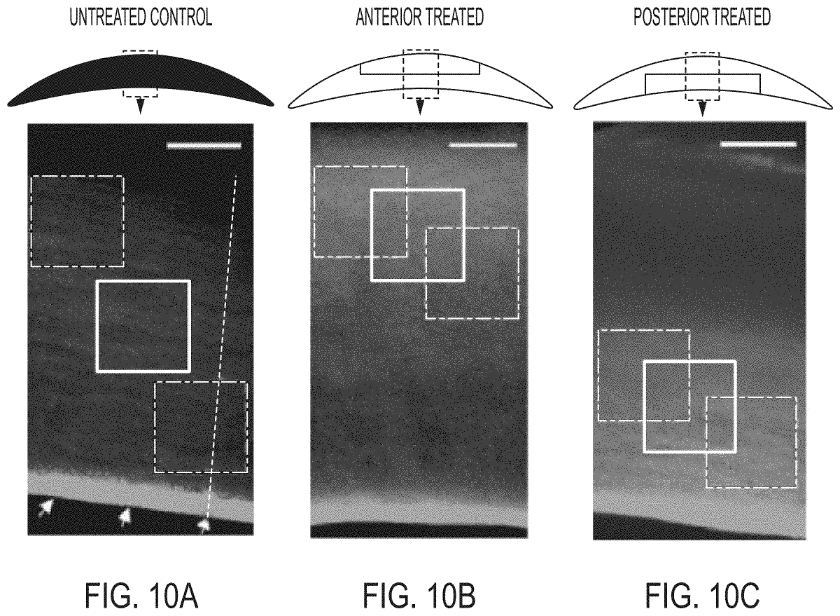

Referring now to FIGS. 10A-C, cross-linking can be spatially resolved to particular layers of the cornea. For example, cross-linking can be limited to an anterior or posterior layer of the cornea. The layers can be defined as within a specified distance of an anterior or posterior surface of the cornea, respectively, e.g., the posterior surface of the cornea epithelium. Exemplary layer thicknesses include: about 50 microns, about 100 microns, about 150 microns, about 200 microns, about 250 microns, and the like. This thickness can be measured from the apex of either the anterior or posterior corneal surfaces, both of which are curved. The posterior layer can include the corneal stroma at the center and/or the periphery of the treatment layer.

The treatment can be carried out on multiple treatment layers that are adjacent to each other, or on treatment layers that are not adjacent to each other. For example, treatment can be carried out starting at the anterior surface of the cornea and proceeding to additional layers below, toward the posterior of the cornea. Not all adjacent layers might be treated, and some layers may be skipped to achieve a desired physical effect in the cornea. For example, only layers at or near the anterior surface of the cornea may be treated, or only layers at or near the posterior surface of the cornea may be treated, or both of the above sets of layers, leaving the central layers of the cornea untreated.

Exemplary Cross-Linking Patterns

Light energy pulses can be applied in a variety of patterns to produce a desired corneal treatment. For example, the curvature of the cornea can be modified to change the refractive power of the cornea. The applied pattern can be a custom-generated pattern based on imaging of a particular subject's cornea. However, and without being bound by theory, Applicant describes general principles of cross-linking patterns below.

Corneal curvature can be flattened to reduce the optical power of the cornea by cross-linking in a solid pattern that extends over the center of an iris posterior to the cornea. Corneal curvature can be steepened to increase the optical power of the cornea by cross-linking in a pattern surrounding, but not extending over the center of an iris posterior to the cornea. For example, an un-cross-linked region over the center of the iris can have a cross-sectional dimension of about 4 mm.

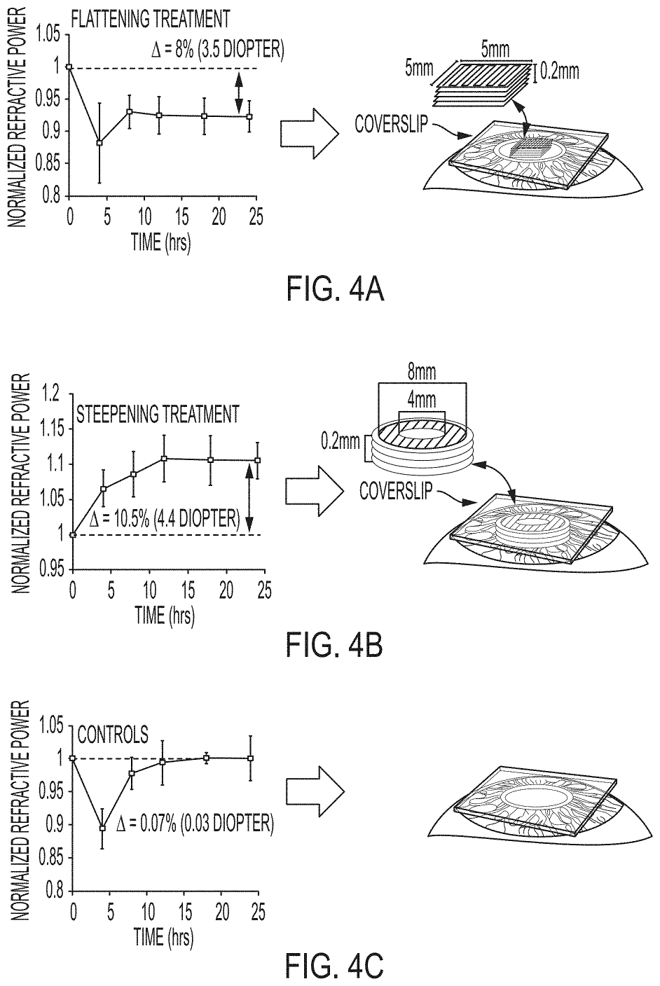

Although a square and an annular pattern are depicted in FIGS. 4A-B, respectively, other shapes can be utilized. For example, the cross-linked region and the un-cross-linked region (if any) can approximate a variety of shapes such as circles, ellipses, triangles, quadrilaterals, rectangles, squares, squircles, trapezoids, parallelograms, rhombuses, pentagons, hexagons, heptagons, octagons, nonagons, decagons, n-gons, and the like.

Additionally, cross-linking within a pattern can be produced using various sub-patterns within the outline of the pattern. For example, cross-linking can be performed in rows and/or columns that begin and break at the borders of the pattern. In some embodiments, cross-linking can wrap in a zigzag or serpentine manner from row-to-row. In still other embodiments, the pattern can be a matrix of cross-linked dots (e.g., in a rectangular grid or close-packed pattern). In still other embodiments, cross-linking can occur in lines that follow the pattern. For example, the pulses can form an annulus or spiral.

Also, cross-linking can be performed in multiple overlapping planes within a corneal layer. For example, a plurality of planes (e.g., 2, 3, 4, 5, and the like) can be cross-linked at a depth offset of about 25 μm, about 50 μm, and the like.

Without being bound by theory, it is believed that a substantially linear refractive power change based on number of treatment layers will be achieved until saturation is reached due to the finite thickness of the cornea. In an embodiment, an 8 diopter refractive power change was achieved by using 15 treatment planes.

As used herein, “planes” (or “layers”) can either include the classical geometrical definition as a flat, two-dimensional surface or can refer to a treatment surface having a defined depth from a curved surface or a treatment layer. (In some embodiments, the application of a cover slip to the cornea will flatten or substantially flatten the normally curved cornea.)

Depths can be determined by measuring thickness of the cornea with a pachymeter, then focusing the light energy pulses on desired locations within the cornea.

Exemplary Systems

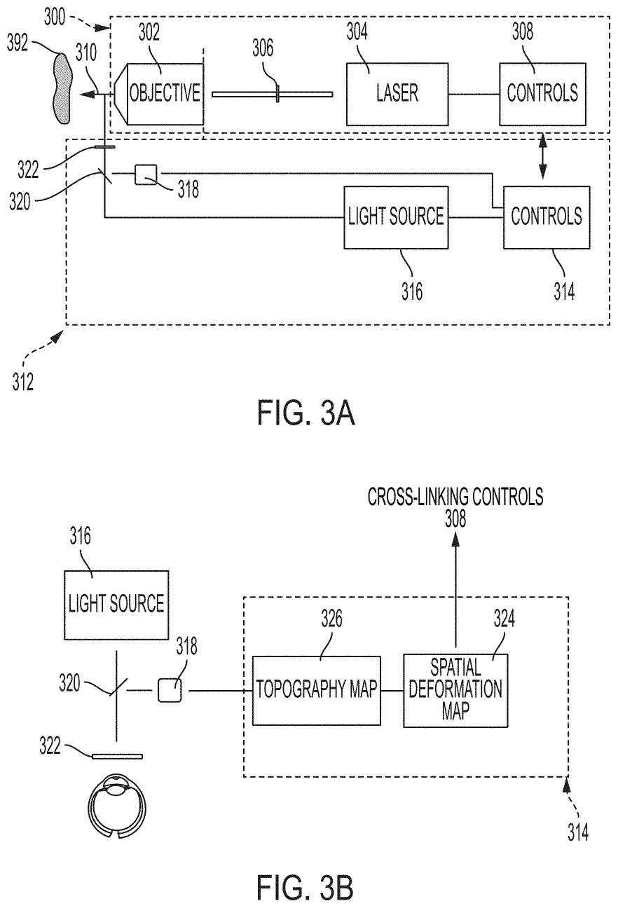

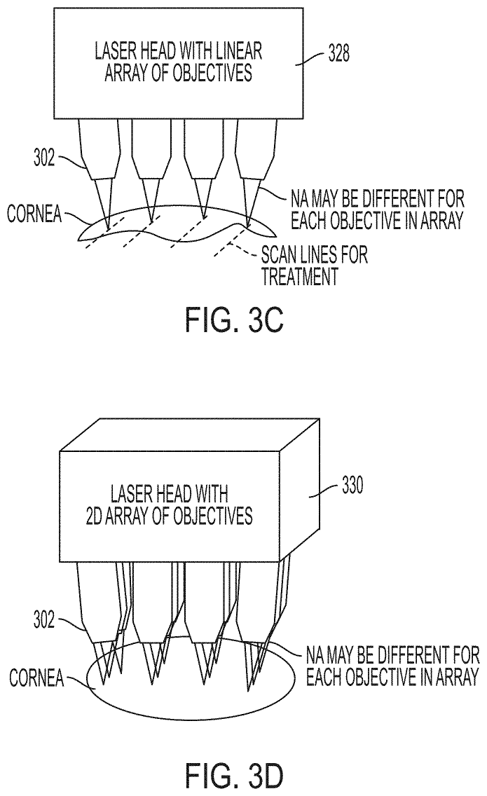

As shown in FIG. 3A, an embodiment of the cross-linking system 300 includes an objective 302. The objective can be high-magnification lens (e.g., 40×).

The objective 302 can be a scanning objective with a large numerical aperture. The large numerical aperture (NA) allows the objective 302 to focus diffuse light to a small area. A laser 304 supplies the light (e.g., laser light) to the objective 302. In one embodiment, the NA is 0.4. In another embodiment, the numerical aperture is 0.6, with a long working distance. However, the NA could be varied together with the pulse energy to achieve similar effect in a different control volume. Without being bound by theory, Applicant believes that NAs below 0.4, between about 0.4 and about 0.95, above 0.95, and above 1 would be capable of creating low-density plasma without causing optical breakdown.

In an embodiment, one or more optical filters 306 can be interspersed between the laser 304 and the objective 302.

The laser 304 can be a femtosecond laser that outputs laser light. In some embodiments, the laser light has a single frequency, and in other embodiments includes multiple frequencies. Embodiments can use any wavelength including multiple or continuous spectra covering a wide range of wavelengths. In embodiments, radiation at frequencies that may harm tissue or reduce the locality of the generation of reactive species are minimized or eliminated. Radiation that may be directly absorbed by the collagen can be minimized or eliminated, e.g., through filters. In an embodiment, the frequency or frequencies of the laser 304 are outside of the ultraviolet range. In embodiments, the frequency or frequencies of the laser 304 are in the infrared frequency band. The laser 304 receives control input from controls 308, which can be implemented on a stand-alone processing device, e.g., a computer executing software, or as embedded circuitry of the system.

Generation of such short pulses can be achieved with the technique of passive mode locking. The laser 304 can be any suitable laser type, including bulk lasers, fiber lasers, dye lasers, semiconductor lasers, and other types of lasers. In an embodiment, the laser operates in the infrared frequency range. In other embodiments, the lasers may cover a wide range of spectra domain. In embodiments, the disclosed subject matter can be implemented as an add-on system to a femtosecond laser system, such as used in certain Lasik systems.

In particular embodiments, the laser can be a Nd:Glass femtosecond laser. In embodiments, the laser wavelength can be in the range from about 250 nm to about 1600 nm. In embodiments, the femtosecond laser can have a temporal pulse width of from about 20 fs to about 26 ps (picoseconds). In embodiments, the pulse energy is from about 0.1 nJ to 100 nJ, 0.1 nJ to about 50 μJ, 0.1 nJ to about 10 μJ, from about 0.5 nJ to 50 nJ, or from about 1 nJ to 10 nJ. In embodiments, the femtosecond laser can be a Spirit® femtosecond laser in combination with a Spirit-NOPA® amplifier (Spectra-Physics, Santa Clara, Calif.).

As further shown in FIG. 3A, the objective 302 focuses incoming laser light into a focused beam 310 that irradiates a target. In the example of FIG. 3, the target is corneal tissue 392. The objective 302 may have a large numerical aperture.

Referring still to FIG. 3A, a topography system 312 includes controls 314, which can communicate with controls 308 of the cross-linking system 300. The topography system 312 can include a light source 316 and an imaging device 318, such as a camera. The light source 316 projects light to mirror 320 and a device, such as a mask, to produce an illumination pattern 322. The illumination pattern 322 guides the cross-linking system 300 to induce cross-linking in specified locations to produce the desired change in the treated tissue.

Referring to FIG. 3B, additional details of the controls 314 of the topography system 312 are shown. A spatial deformation map 324 spatially defines the deformation of the cornea, which, when considered with the topography map 326 of the cornea, provides information on where to induce cross-linking.

In embodiments, multiple beams can be provided by splitting a laser beam to multiple scanning objectives. For example, a laser head can include multiple scanning objectives bundled together, as shown in FIGS. 3C and 3D. FIG. 3C illustrates an example of a linear array 328 of objectives 302. FIG. 3D illustrates a two-dimensional array 330 of objectives 302. Although the objectives 302 are illustrated as identical in the drawings, in embodiments different objectives are used at different positions in the array. A high-energy laser beam (e.g., having a pulse energy of greater than about 10 μJ) can be split using a beam splitter to send individual laser beams to each scanning objective. Therefore, the number of passes required to fully treat the cornea can be reduced by providing multiple laser beams simultaneously. In embodiments, an entire corneal layer could be treated simultaneously, e.g., by bundling many scanning objectives to the laser head such that only one pass is required. Beams can treat different x-y coordinates and/or can treat different treatment layers simultaneously.

Implementation in Computer-Readable Media and/or Hardware

The methods described herein can be readily implemented, in whole or in part, in software that can be stored in computer-readable media for execution by a computer processor. For example, the computer-readable media can be volatile memory (e.g., random access memory and the like) non-volatile memory (e.g., read-only memory, hard disks, floppy disks, magnetic tape, optical discs, paper tape, punch cards, and the like).

Additionally or alternatively, the methods described herein can be implemented in computer hardware such as an application-specific integrated circuit (ASIC).

### WORKING EXAMPLES

### Working Example 1

### Cross-Linking of Ex Vivo Porcine Eyes

A total of 60 fresh pig eyes were used for the study. Fifteen of these eyes underwent corneal flattening, and the treated eyes were paired with 10 control eyes. Thirteen eyes underwent laser irradiation to induce post-treatment steepening; these eyes were also paired with 10 control eyes. The remaining 12 eyes were used for a separate control study, to evaluate the effects of the experimental setup.

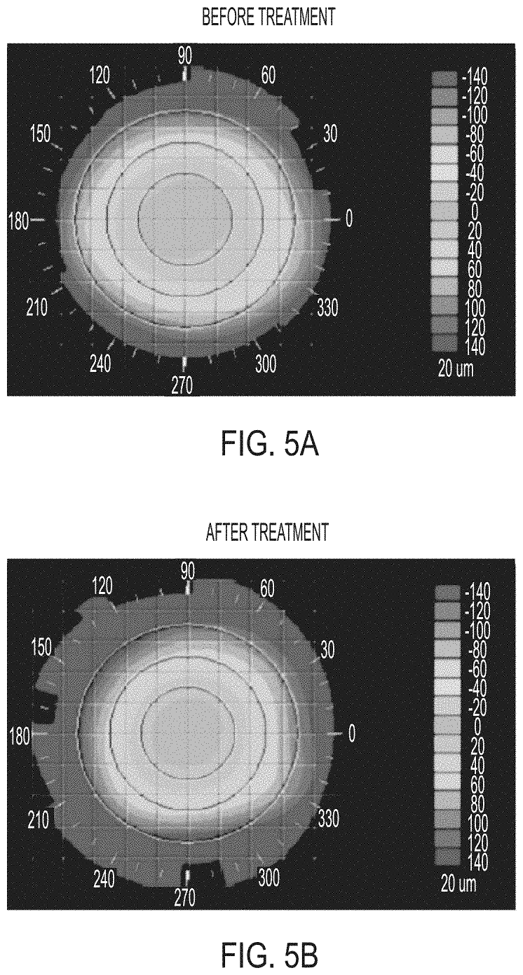

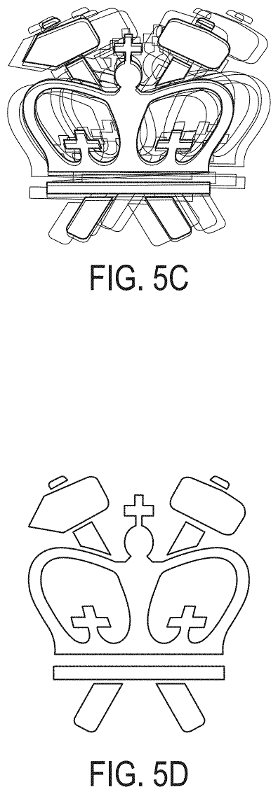

For the flattening treatment (FIG. 4A), a square in the middle of the eye was treated. A strong change in corneal curvature, corresponding to a change in refractive power of about 12% (about 5.11 diopters on average), was initially observed, followed by partial recovery. Most of the change in curvature occurred within eight hours of treatment, after which the cornea stabilized at a refractive power about 92% the initial level (about 3.45 diopters on average). This significant change became evident when corneal topography before and after treatment was paired with the corresponding virtual vision, demonstrating the effects of the correction of refractive errors applied (FIGS. 5A-D).

The initially large change in refractive power is due to a combination of the effects of the treatment itself and experimental conditions, which include temporary flattening of the cornea with a coverslip to ensure even volumetric exposure of the stroma to laser irradiation. The coverslip has an effect analogous to that of orthokeratology (ortho-K), a temporary reshaping of the cornea used to reduce refractive errors, and the duration of the effect is similar to that of an ortho-K procedure.

Once the coverslip effect wears off, the adjusted curvature remains stable throughout the rest of the 24-hour period. By contrast, laser treatment of the peripheral zone of the cornea leads to its steepening (FIG. 4B). The effective refractive power of pig eyes is significantly increased by treatment of a ring-shaped region. In the case of corneal steepening, the effective power of the eye increases gradually over a 12-hour period, after which it stabilizes at a new value higher than that before treatment. This indicates that the induction of new CxLs counteracts the influence of the cover slip. For confirmation that the induced changes were photochemical in nature, with no influence of the thermal denaturation of collagen fibrils, Applicant measured the laser-induced changes in corneal temperature. The relative change in temperature at the focal volume and in its immediate vicinity was less than 7° C. The heating induced by the treatment was, therefore, well below the threshold for the thermal denaturation of collagen. Furthermore, light microscopy with a microscope equipped with Nomarski interference contrast optics revealed no difference in refractive index between the treated and untreated parts of the cornea, consistent with an absence of corneal hazing.

### Working Example 2

### Spatially Resolved Alterations of Rabbit Eyes Ex Vivo

Ex vivo rabbit eyes for the experiments were delivered to the lab as intact rabbit heads from a local abattoir (La Granja Live Poultry Corporation, New York, N.Y.) within an hour after being euthanized. Eyes were isolated, rinsed with Dulbecco's phosphate-buffered saline (DPBS, 1×, Sigma-Aldrich), inspected for presence of defects and gradually brought to room temperature in a humidified chamber. Defective samples were discarded. After removing excess tissue, the eye globe was mounted onto a custom-built eye holder (FIG. 6). In order to maintain the eye pressure (−16 mm Hg), an intravenous (IV) system filled with the 0.9% sodium chloride solution (Hospira Inc.) was attached to the eyeball via 22G injection needle (BD). A customized digital pressure gauge (OMEGA™ PX154) was applied to adjust the pressure level. Corneal thickness was measured by a DGH™ PACHETTE™ 2 Pachymeter (DGH Technology, Inc.). Before treatment, the corneal surface was covered with a microscope cover glass (#1 Microscope Cover Glasses, VWR) to ensure even volumetric treatment of the cornea and reduce light scattering.

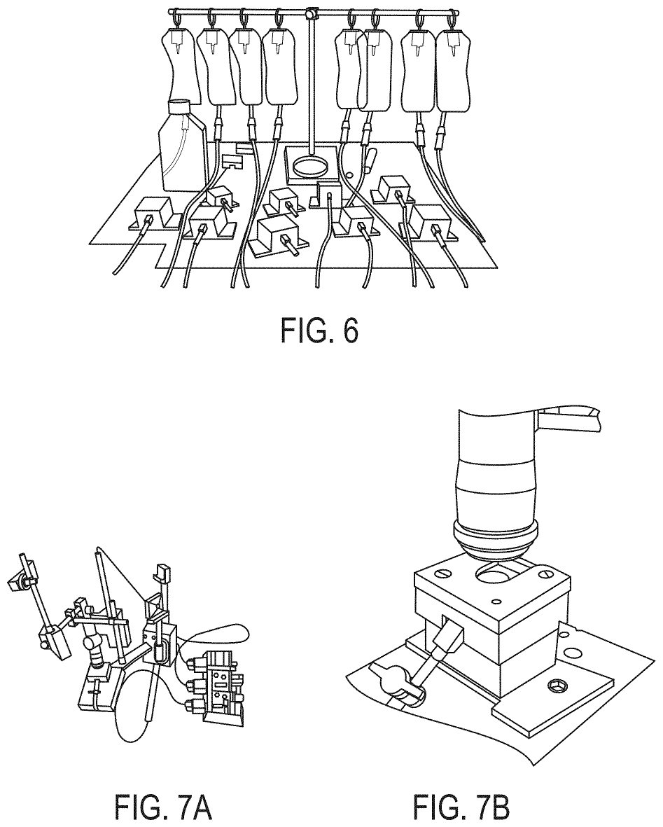

A Nd:Glass femtosecond laser oscillator system (HIGH Q LASER™, Spectra-Physics) was applied to generate laser pulses with temporal pulse width of 99 fs and 52.06 MHz repetition rate at 1060 nm wavelength. A ZEISS® PLAN-NEOFLUAR® 40×/0.6 objective lens was employed to focus the beam, and the average pulse energy and photon energy produced by the proposed lasing system are 60 mW and 1.1696 eV respectively after the objective lens. The laser beam was motorized by Z825B motors (Thorlabs) through a 3-dimensional PT1 translation stage (Thorlabs). Schematic diagram shows a femtosecond laser optical system set up in FIGS. 7A and 7B. The laser beam was initially focused on the superficial surface of the cornea. Laser pulses were delivered within a 25 mm² square via zigzag motion of the focused beam at 2.2 mm/s feedrate. Multiple planes parallel to the corneal surface were treated with 50 μm distance between two consecutive planes.

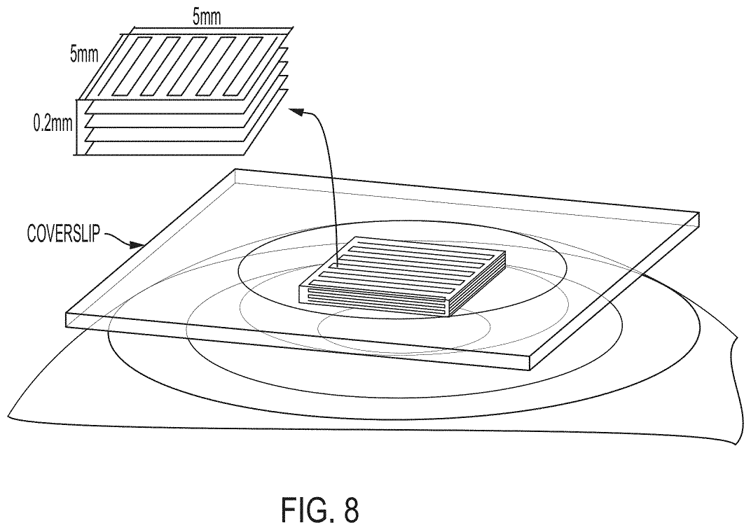

In any of the disclosed embodiments, the lasing trajectory may follow a zig-zag pattern within the treatment layer, with the focal volume moving in the horizontal plane at 30 mm/s. Schematic diagrams of treatment paths are shown in FIG. 8. Two treatment patterns were applied in the study to show the selective spatial treatment ability. The anterior treatment pattern utilized the treatment from the superficial surface to the central cornea. The posterior treatment pattern employed the treatment from the central cornea to the endothelium layer by applying a subtraction of the corneal. A paired control eye was placed on the same stage for every treatment. After the treatment, the cover glass was carefully removed. Topographic refractive power measurement of an entire corneal area by an EYESYS® VISTA™ non-contact eye-topographer (EyeSys Vision Inc.) was performed before and every 3 hours during the 24 hours period after the application of the laser treatment, to assess the effect of laser light-induced corneal crosslinking and reshaping. After topographic characterization the corneal tissue was isolated form eye globe and prepared for confocal imaging and two photon autofluorescence (TPF) imaging. The cornea, retina, and lens were isolated, fixed with 10% formalin overnight and desorbed with 70% ethanol for 24 hours prior to histology staining. Histology staining was performed by Columbia Medical Center Histology Service. Briefly, samples were embedded in paraffin wax and cut into 5 thickness slices through cross section and stained with hematoxylin and eosin. Histological slices were imaged by a VHX 5000 digital microscope (Keyence Corporation, N.J.) and processed by IMAGEJ™ software.

Confocal Laser Scanning Microscopy (CLSM)

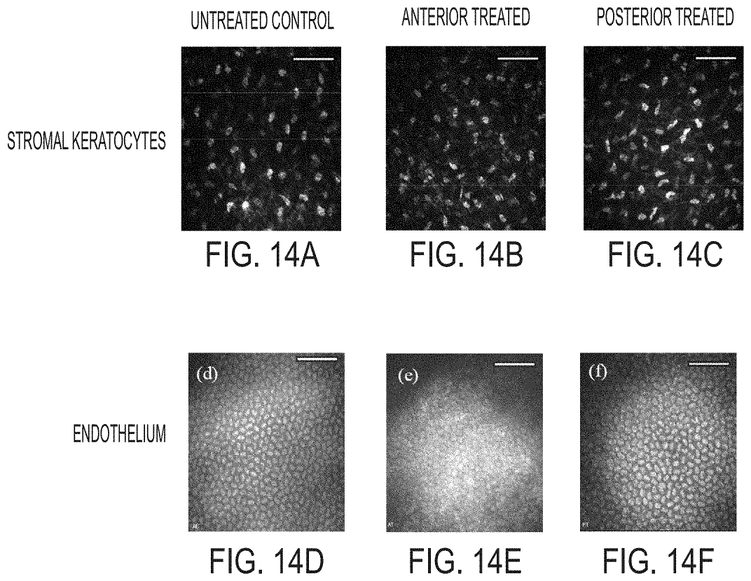

Referring now to FIGS. 14A-14F, CLSM was employed for cellular evaluations of corneal tissues. CLSM imaging was performed with the HRT3-RCM laser scanning system (670 nm laser beam, Heidelberg Engineering) equipped with a 63×/0.95 NA water immersion objective (Zeiss). A disposable sterile plastic cap was placed on the objective to maintain the distance between the corneal surface and the objective. GENTEAL™ water-based gel was applied as a coupling medium. Imaging was characterized before and immediately after the laser irradiation. The entire corneal volume was scanned and recorded, with optical sections through the epithelium, stroma, and endothelium.

The images show no evidence of negative effects of the applied femtosecond laser treatment on the cellular component of the rabbit cornea. In the cases of the anterior and the posterior application of the treatment, CLSM shows no significant differences in the morphology or cellular density of the stromal keratocytes and endothelium layers comparing to the untreated control. These preliminary results could contribute to the evidence of safe application of the tested laser irradiation for vision corrections.

Two-Photon Fluorescence (TPF) Microscopy

Isolated untreated control and laser treated corneal samples were cut into 2 mm² blocks by a customized slicer and mounted by 50% glycerol in PBS in a 3 mm Petri dish filled with PBS solution. TPF was conducted by a two-photon microscope (Bruker) with MAI TAI™ DEEPSEE™ Ti: Sapphire laser (Spectra Physics) as the excitation source. A 40×/0.8 NA water immersion objective (Olympus) was applied to collect the fluorescence signal. The signal was registered with two different photomultiplier tubes, one in the red (580-620 nm) and one in the green (480-570 nm) wavelength regime. Excitation wavelengths used were 826 nm to excite collagen matrix

Results

Due to the nature of the delivery of concentrated nonlinear laser energy, the alteration of refractive power is spatially resolved and, thus, controllable. This may be particularly applied for the treatment of selective volumetric regions of corneal tissue that yields macroscopic changes in overall corneal curvature, which can be utilized for selectively treatment of myopia, hyperopia, stigmatism and irregular astigmatism. In order to show the spatial resolution of the proposed treatment, two treatment patterns were employed in this study. The anterior treatment pattern utilized the treatment from the superficial surface to the central cornea, and the posterior treatment pattern applied the treatment from the central cornea to the endothelium layer.

A total of 47 eyes were applied in this study. 20 eyes were subjected to anterior treatment, whereas 8 eyes were exposed to the posterior treatment. Treated samples were properly paired with untreated control eyes. The remaining 8 eyes were used as untreated control eyes to evaluate the experimental setup.

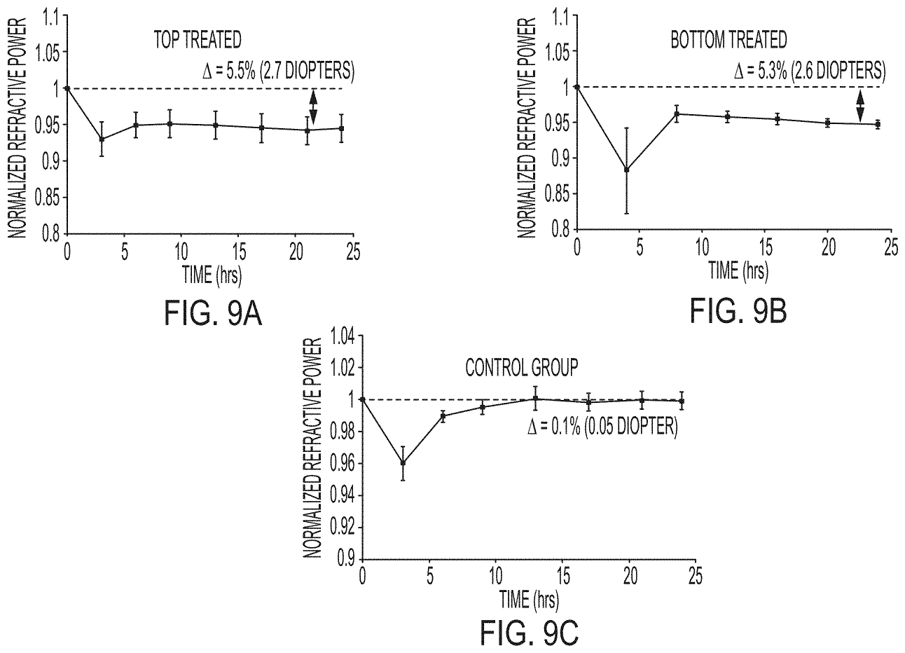

For the anterior treatment pattern, initially a steep change in corneal curvature, corresponding to an approximate 7.1% change in the overall refractive power (about 3.5 diopters on average), is followed by a partial recovery. The major curvature change occurs within 8 hours from the treatment, after which the corneal refractive power stabilizes at about 94.5% of its initial refractive power before the treatment (about 2.7 diopters on average). The relative significant change of corneal refractive power is further evident by the paired untreated control eyes, which showed approximately no change of refractive power over the 24 hours characterization period (FIG. 9C). Initially, the major change in refractive power is attributed to the superposition of the treatment itself and the temporary flattening of the cornea by the application of a cover slip. The duration of the coverslip effect is comparable to the clinical orthokeratology (ortho-K) operation. After the cover slip effect wears off, the adjusted curvature remains stable throughout the remainder of the 24 hours period. The NLO-HRMac imaging of three-dimensional collagen organization of the rabbit cornea showed the bulk of the rabbit cornea exhibits a parallel arrangement of collagen fibers, with collagen intertwining present only in the anterior aspect. Thus, theoretically, the treatment of posterior region and anterior region should not be the same. However, the posterior treatment leads to a similar effect as the anterior treatment by the proposed laser irradiation (FIG. 9B). This may demonstrate that the newly formed CxLs are dominating the overall refractive power adjustment, and the introduction of CxLs by the proposed method may not be dependent on the orientation of corneal collagen. Initially, a steep change in corneal curvature corresponds to an approximate 12% change in refractive power (about 5.7 diopters on average), which is followed by a partial recovery. The most curvature change also occurs within 8 hours from the treatment, after which the cornea stabilizes at about 94.7% of its initial refractive power (about 2.55 diopters on average).

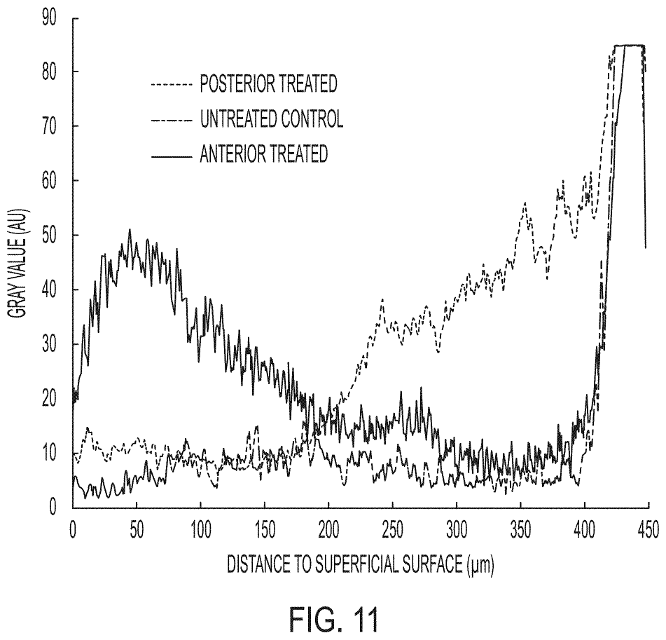

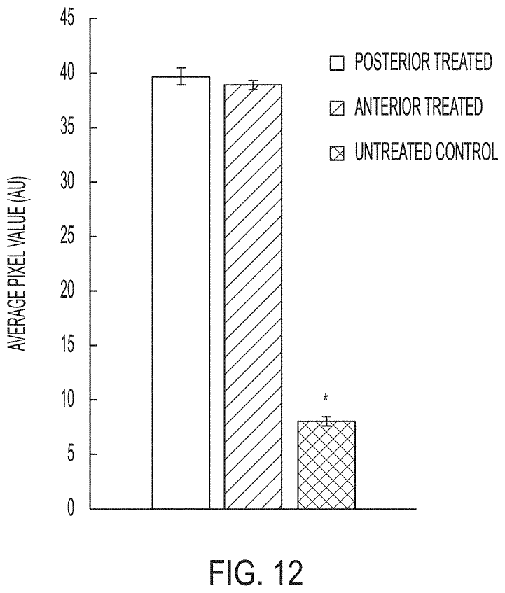

Two-photon autofluorescence (TPF) identifies fibrillar collagen in response to near-infrared laser light excitation. Thus, TPF imaging is employed to evaluate the laser induced CxLs in the cornea. The collagen extracellular structural differences among anterior treated, posterior treated, and untreated control eyes are presented in FIGS. 10A-C. The excitation of tyrosine, dityrosine oxidation products, and pyridinium-type fluorophores are responsible for the contrast of the TPF images. TPF images showed a bright region for both untreated control and laser treated samples near the corneal posterior, which may result from the Descemet's membrane, the basement membrane for the endothelial layer composed mostly of different types of collagen. Similar to the reported TPF images of collagen hydrogels crosslinked by glutaraldehyde and rabbit corneal tissue treated by riboflavin and UVA light to induce corneal CxLs, the treated region of laser irradiated samples showed a significantly stronger signal compared to the untreated region or control cornea, indicated that the proposed treatment introduced an increased CxLs density. Three lines were drawn on both central and peripheral zones through the whole corneal thickness indicated by the arrows in figure FIG. 10A. The averaged gray values of the three lines for the untreated control and laser treated samples are presented in FIG. 11. The average pixel values of histograms for boxed regions in FIGS. 10A-C indicates that the intensities for the laser treated eyes were much stronger than the untreated control eye and the intensities for anterior and posterior treated regions were approximately the same. Corresponding well with the treatment depth, the average gray values showed the size of the treated region were all about 200 μm for both anterior and posterior treated corneas, together with the average pixel values of the histograms, suggesting that the crosslinking efficiency does not diminish as the focusing of the laser pulses is shifted in the cornea. As the treatment pattern goes from anterior to posterior treatment, the collagen-rich zone shifted from anterior to posterior, which revealed the proposed treatment has the spatial selectivity ability.

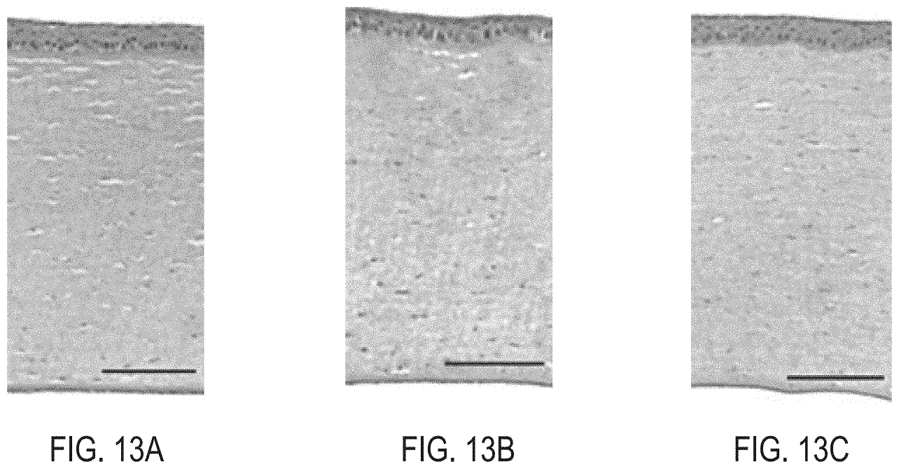

Histological analysis of H&E-stained sections (FIGS. 13A-13C) reveals all main elements of corneal architecture: epithelial and endothelial layers, keratocytes and extracellular stromal matrix. There are no signs of thermal damage such as collagen disorganization, stromal edema, disorganization of cellular components commonly observed in the cases of corneal overheating on the obtained images. Light microscopy shows no differences in corneal structure of the anterior and posterior treated samples comparing to the untreated ones.

Treatment of the posterior stroma provides similar change in corneal curvature to that seen in treatment of the anterior stroma. This is unexpected due to differences in architecture of these two corneal segments, and it goes against conventional wisdom of ophthalmologists. The ability to achieve changes in eye refractive power through treatment of the corneal stroma also allows for treatment to be extended throughout the corneal thickness to treat more severe cases of myopia.

Working Example 3

### Effect of Increasing Treatment Layers on Refractive Power

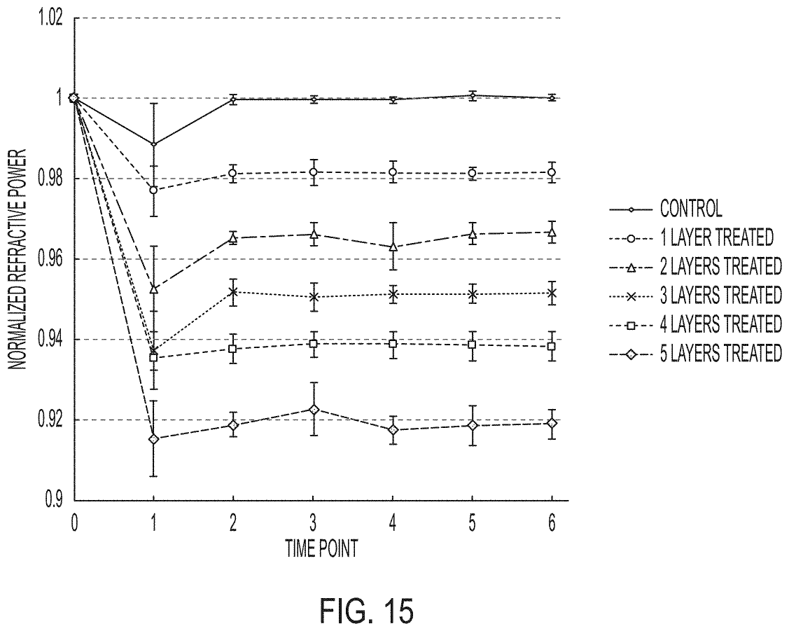

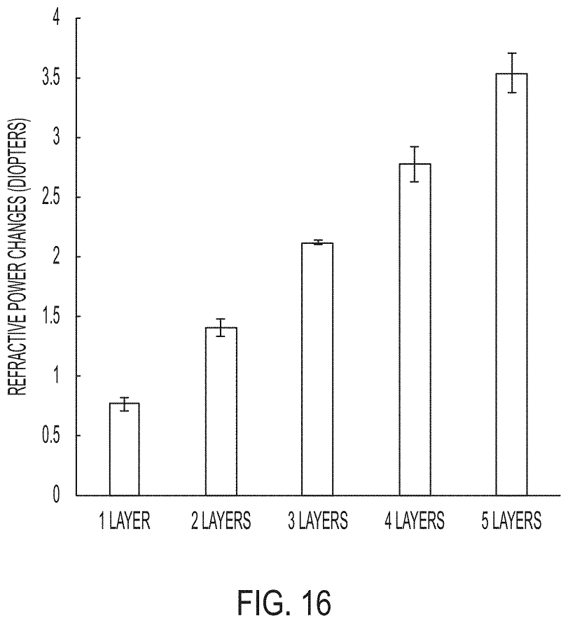

Referring now to FIGS. 15 and 16, ex vivo animal eyes were treated as described herein. The eyes were treated with varying numbers of treatment layers between 0 (control) and 5. FIG. 15 is chart of average normalized refractive power changes for 24 hours with 4 hours between each time point, for 5 control eyes and 4 eyes for each treatment group (a total of 5+4*5=25 eyes). FIG. 16 depicts average diopter changes for each treatment group. Error bars are shown as standard deviation. The refractive power change is remarkably linear with respect to increasing numbers of treatment layers. Based on the measured results and the observed linearity of the refractive power change, an 8 diopter refractive power change would be achieved by using 15 treatment planes. The constraint to the refractive power change is the thickness of the cornea being treated.

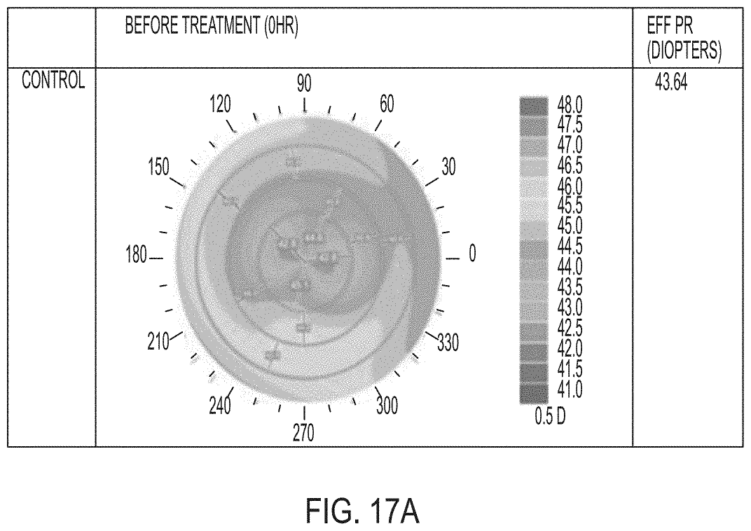

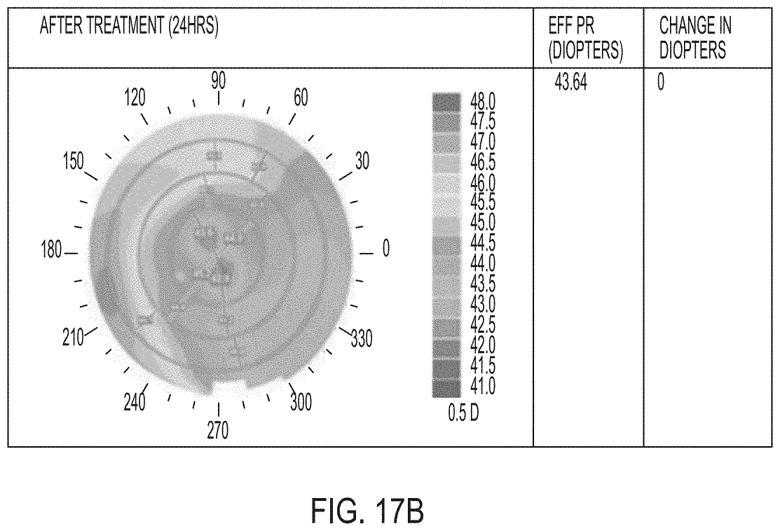

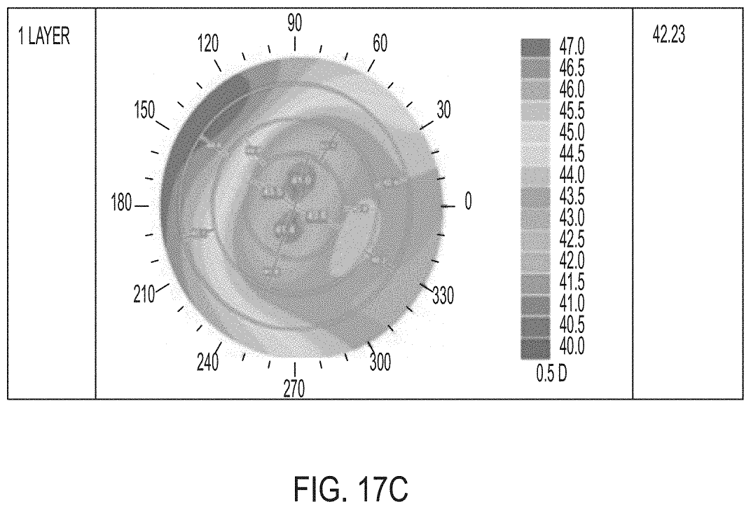

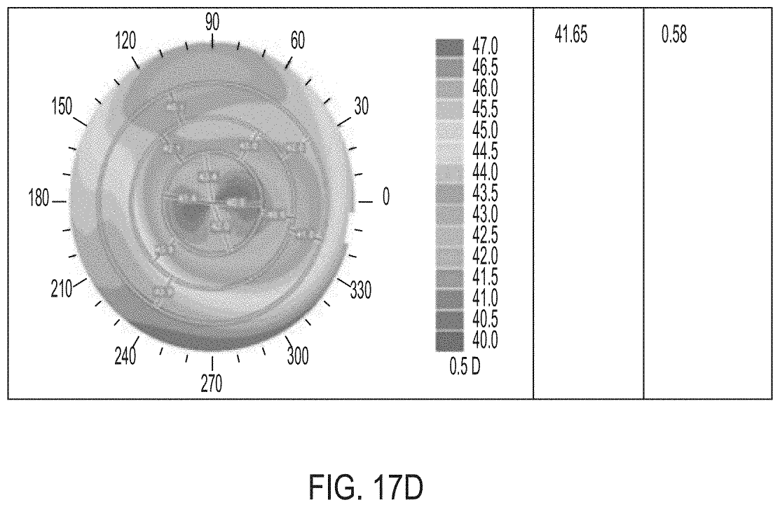

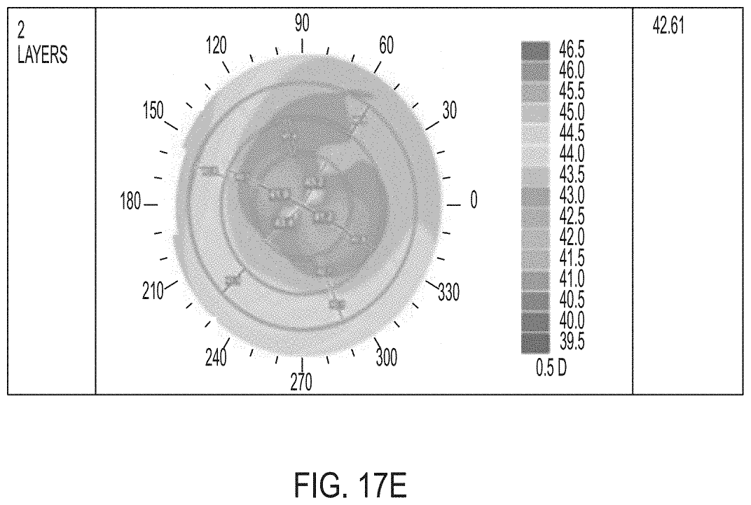

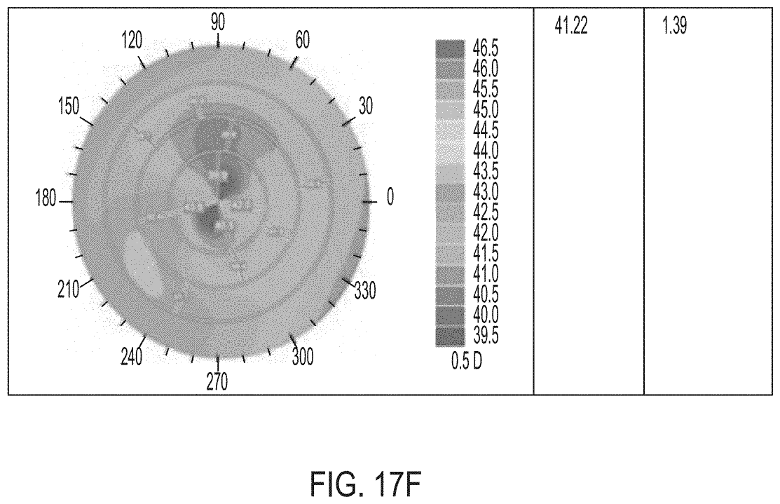

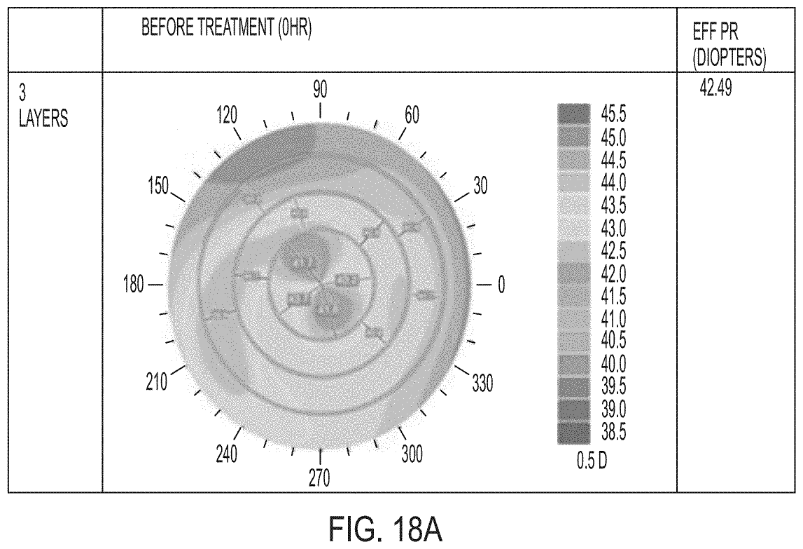

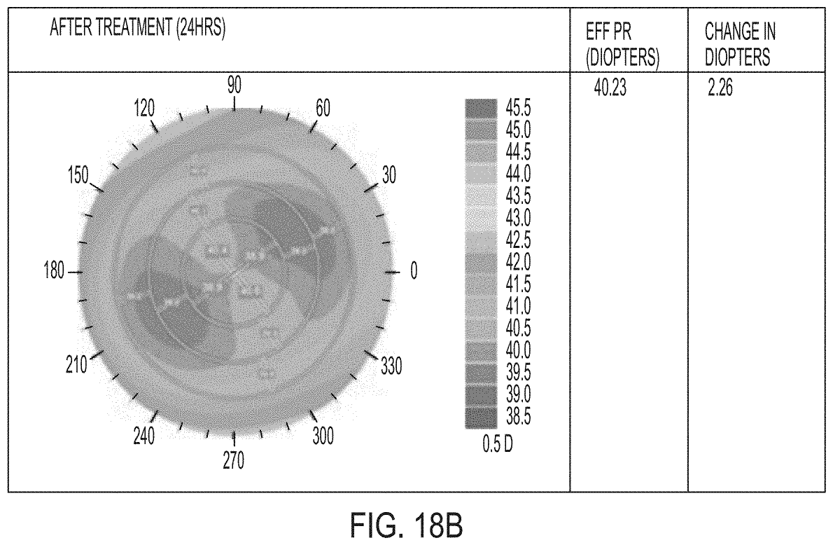

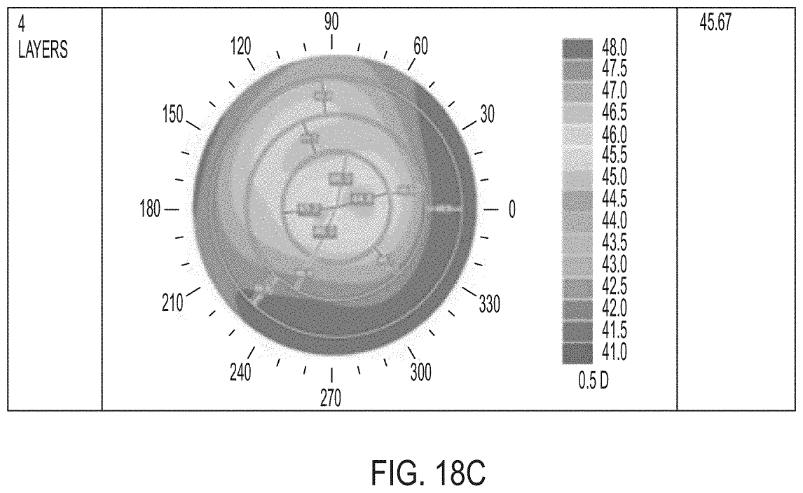

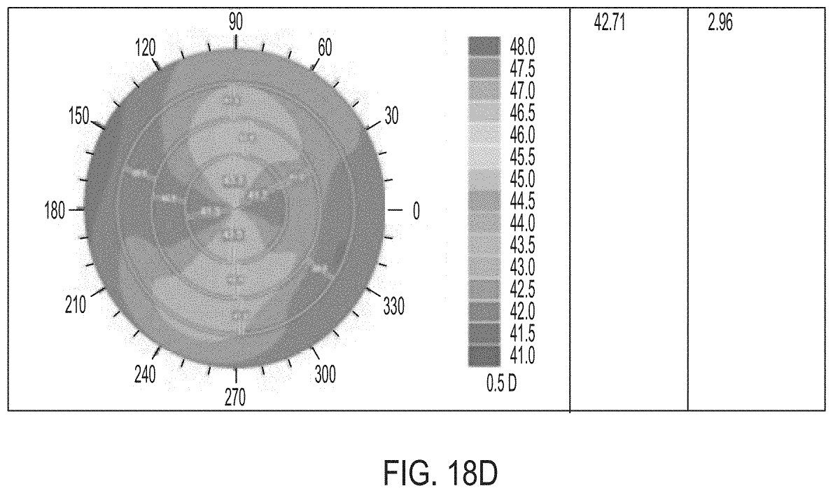

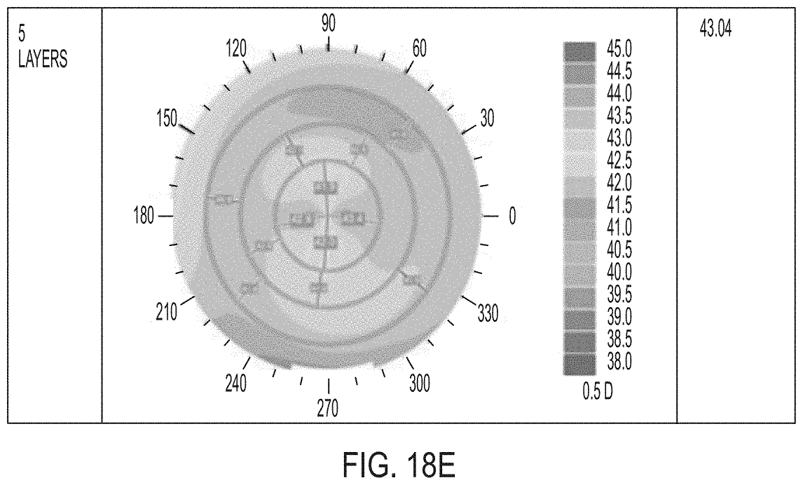

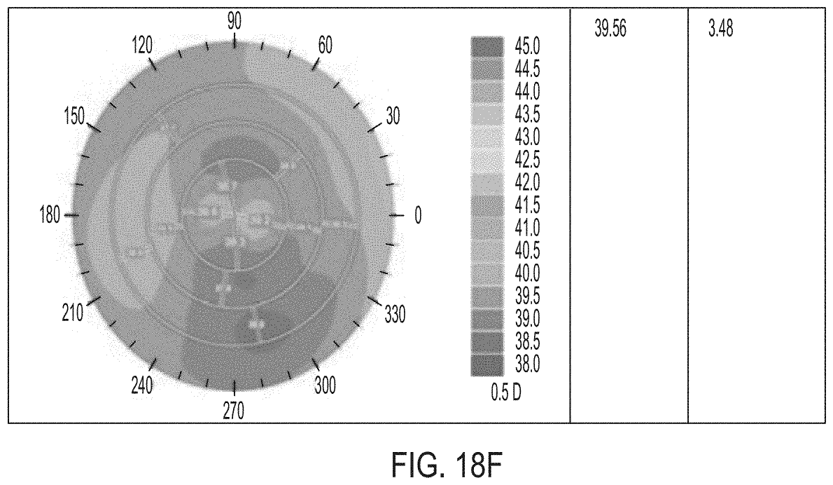

Referring now to FIGS. 17A-F and 18A-F, further results of a controlled parametric study with porcine eyes is shown. The shading within the drawings represents the refractive power. In each of FIGS. 17A-F, the shaded color map illustrates a refractive power map for three eyes used in the test. In FIGS. 17A, 17C, and 17E, the right column provides the EFF PR in diopters. In FIGS. 17B, 17D, and 17F, the right-most column provides the change in diopters as compared to pre-treatment EFF PR after 24 hours. FIGS. 17A-B represent the control eye, FIGS. 17C-D correspond to the eye that was treated with one layer, and FIGS. 17E-F represent the eye that was treated at two layers. FIGS. 18A-B represent the eye that was treated with 3 layers, FIGS. 18C-D represent the eye treated with 4 layers, and FIGS. 18E-F represent the eye treated with 5 layers. While FIGS. 18A, 18C, and 18E represent the EFF PR before treatment, FIGS. 18B, 18D, and 18F illustrate the results 24 hours after the treatment was completed. As is expected, the control eye was not treated and the results show little or no change. The treated eyes show changes in the refractive power represented by the shading in the figures and the included shading key.

Mechanical Properties

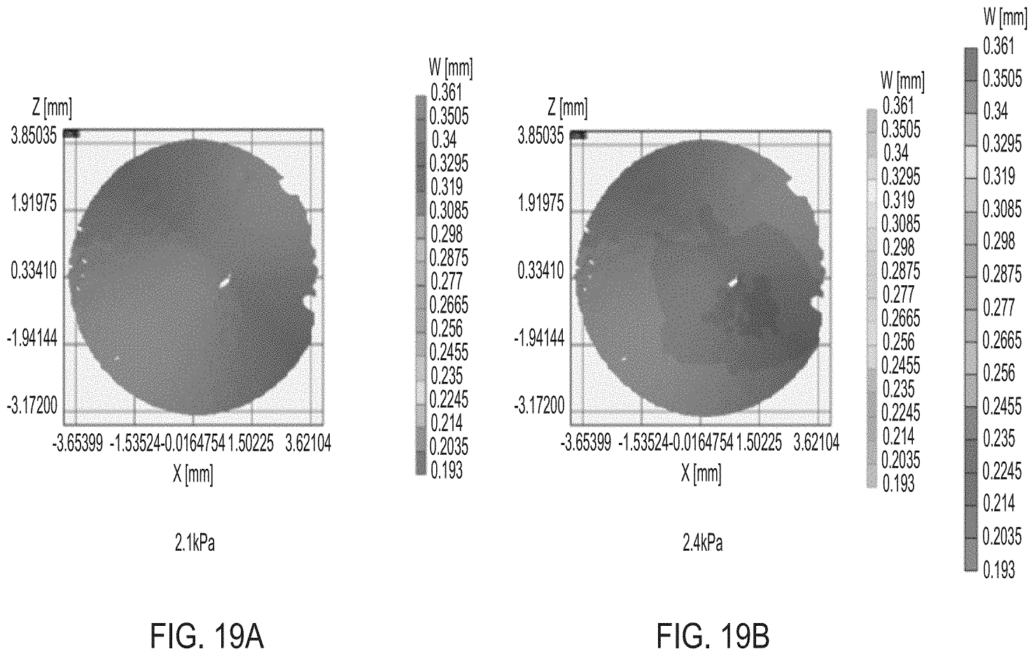

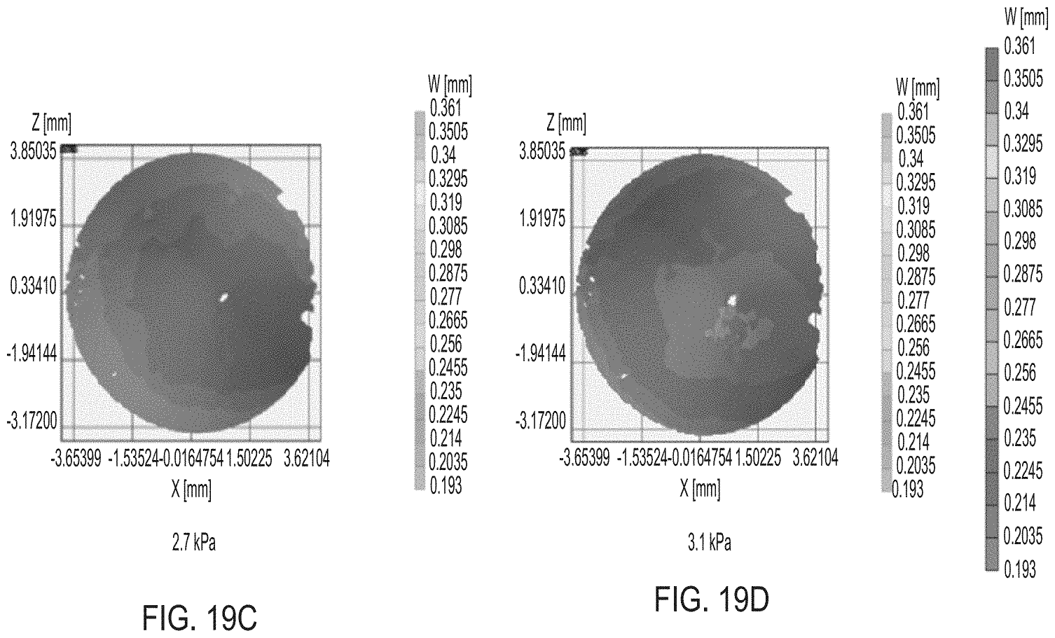

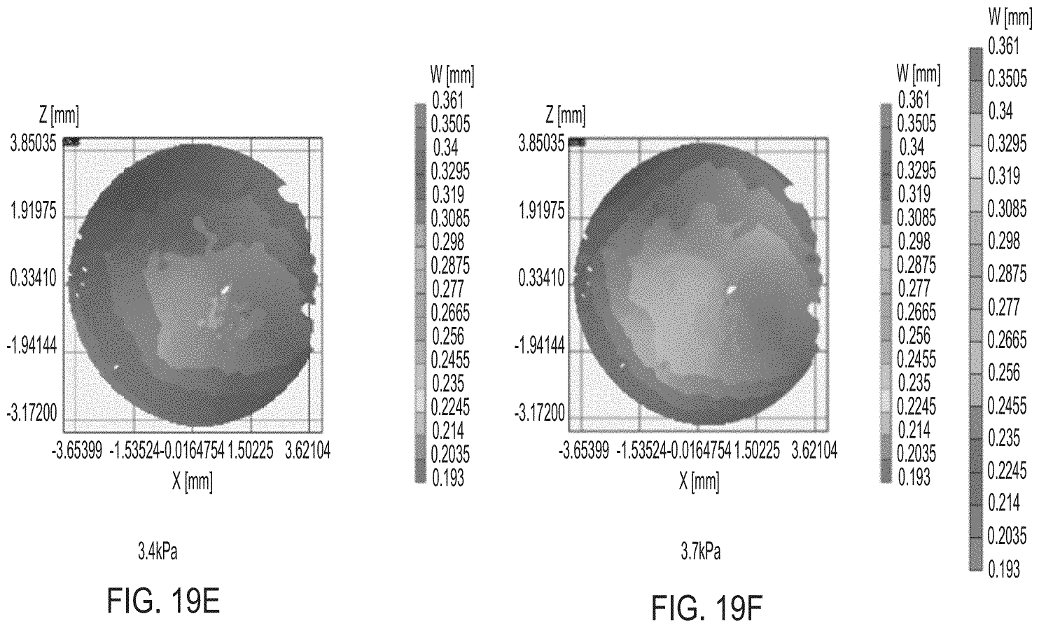

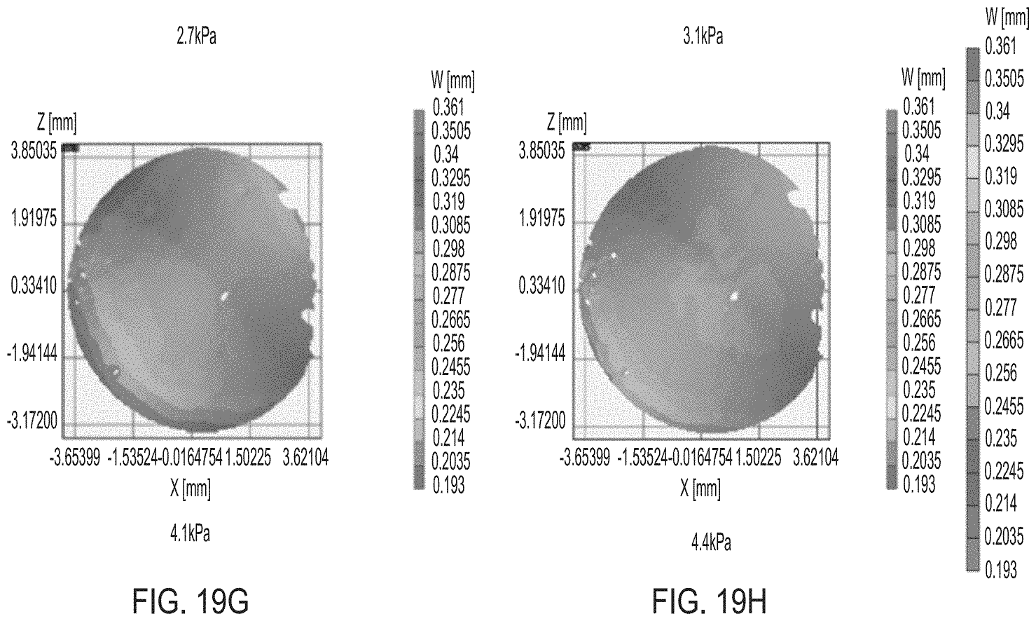

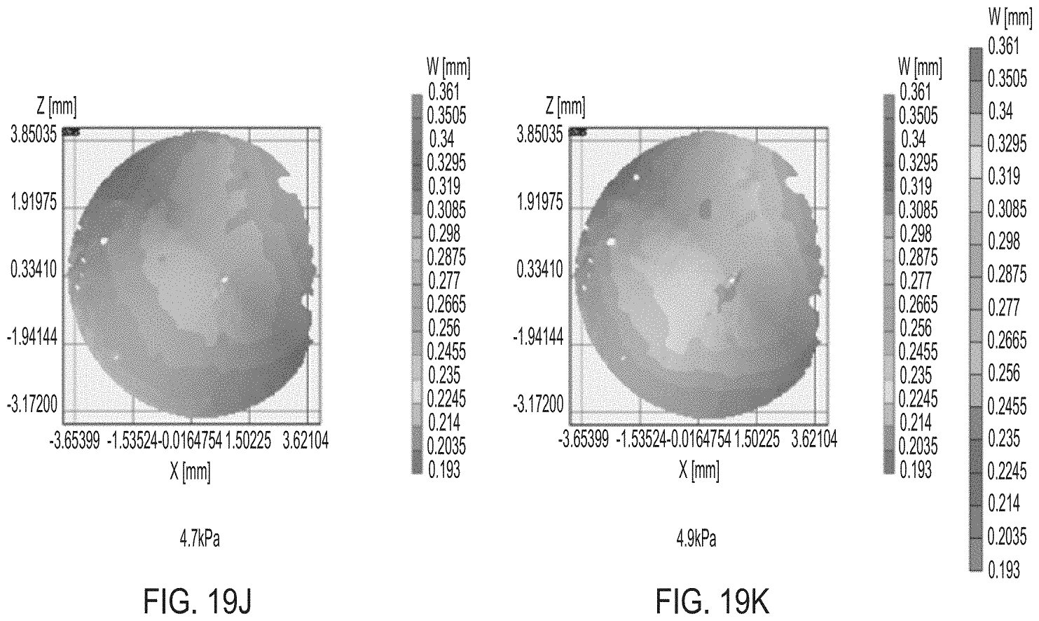

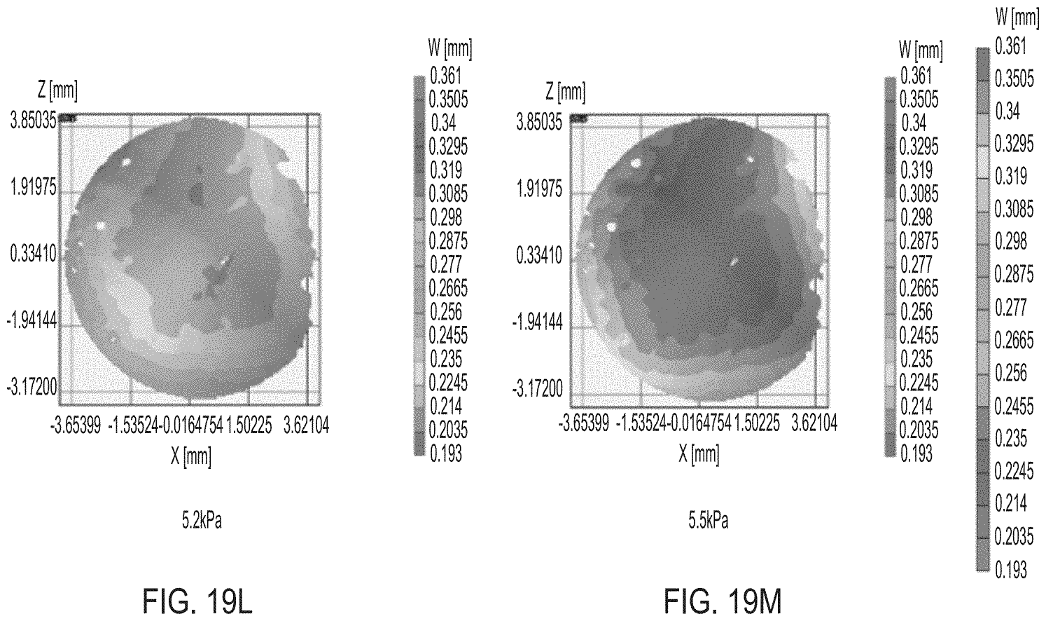

Mechanical properties of corneas were tested before and after treatment at varying layers. Inflation tests provide information about mechanical properties of the cornea. FIGS. 19A-H and 19J-M illustrate displacement maps of the control cornea (i.e., not subject to treatment) in the Z direction when the cornea is subject to different pressures, each listed in the figure in units of kPa. The displacement maps are extracted from the time history of the inflation test.

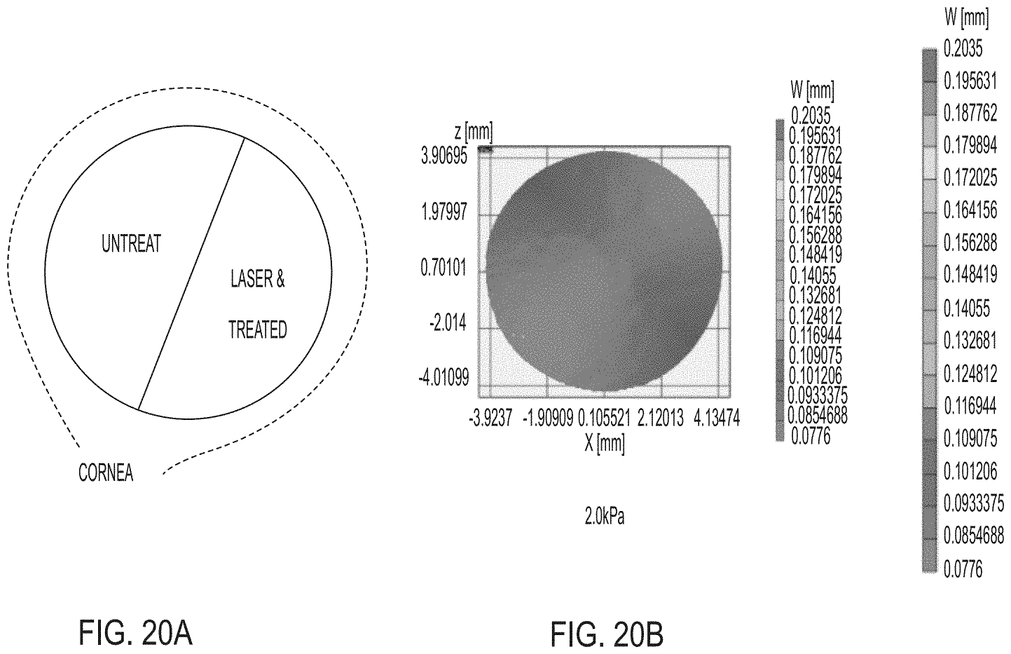

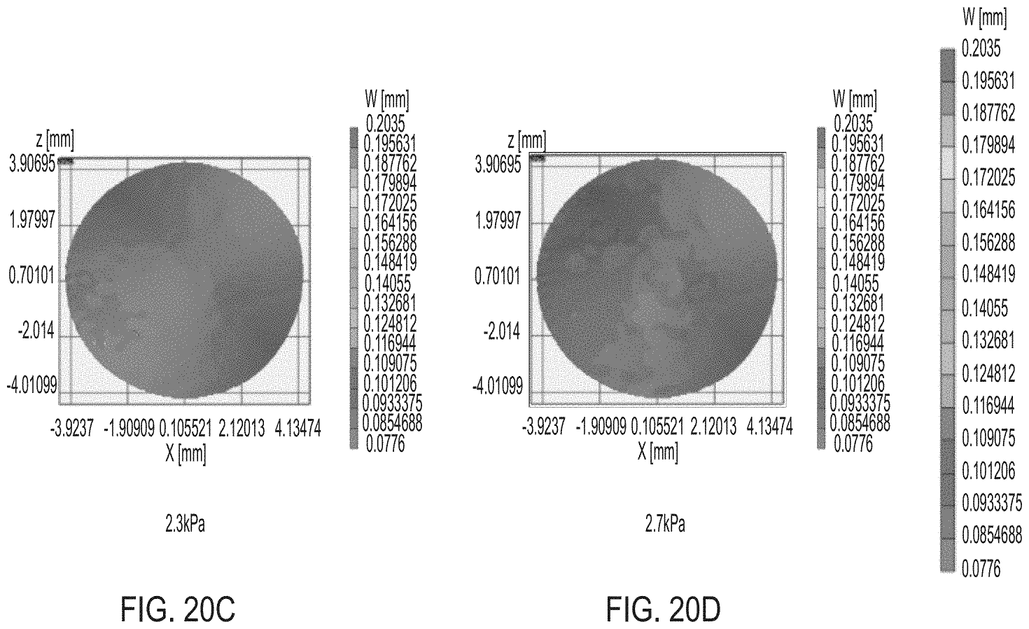

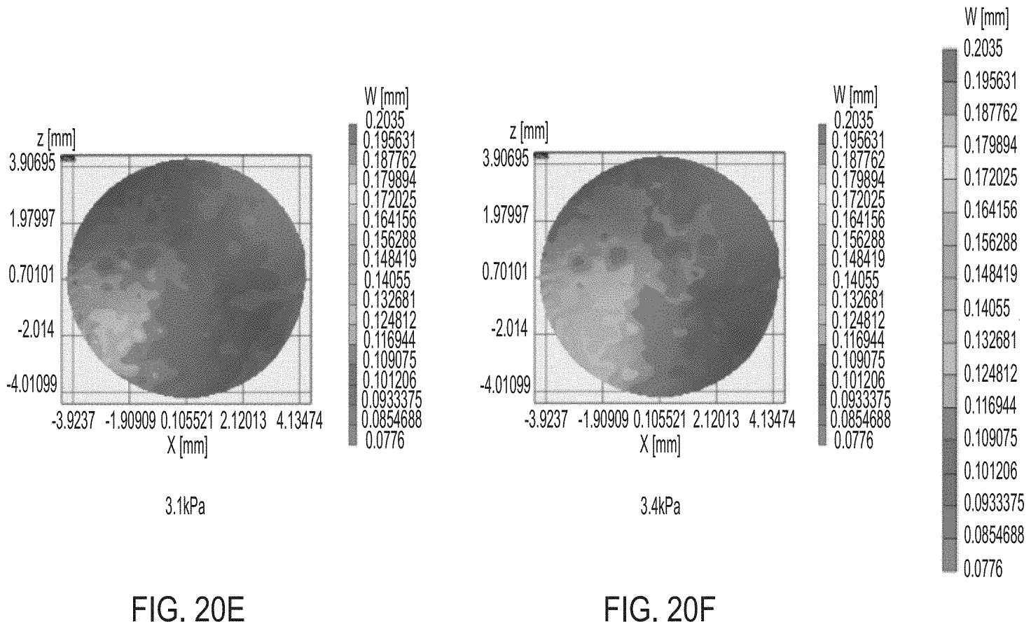

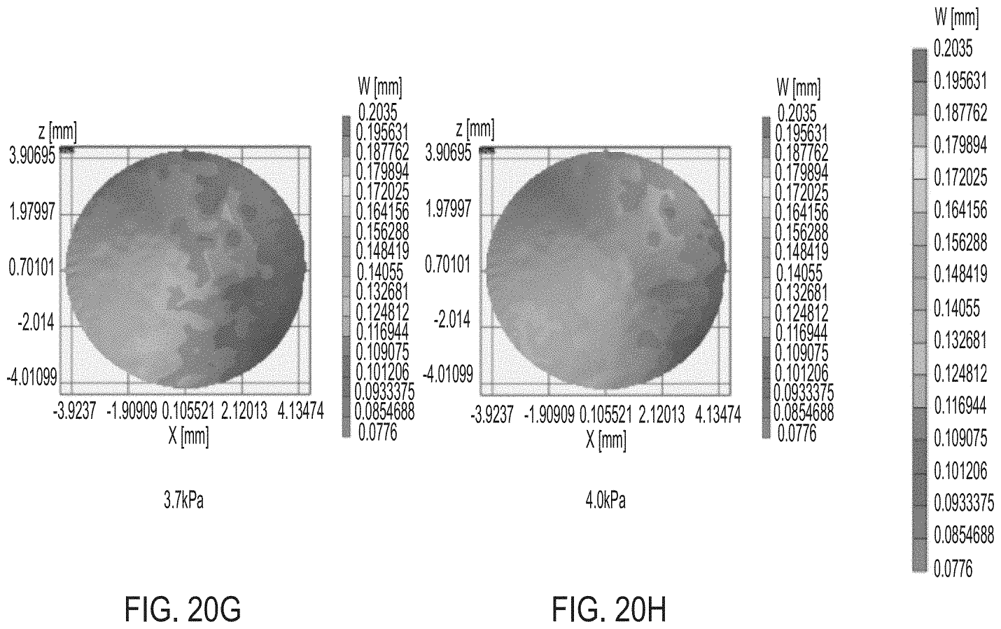

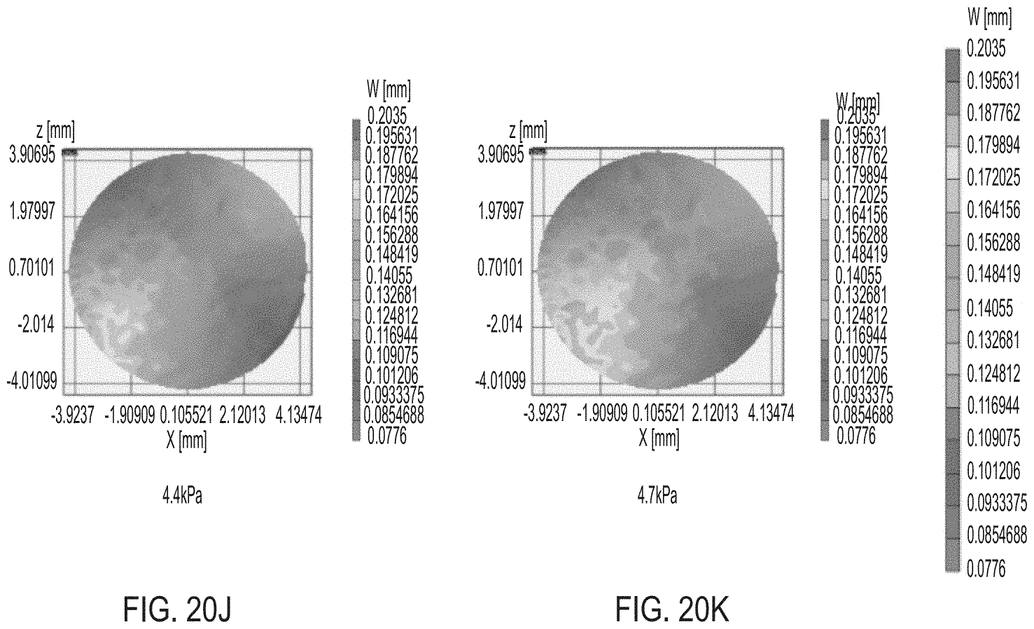

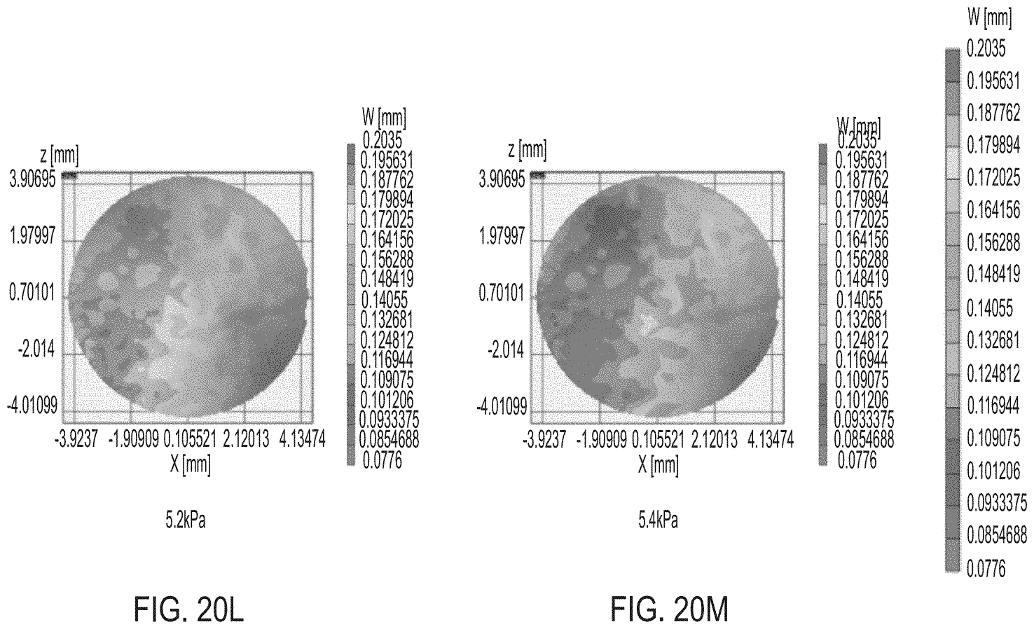

FIG. 20A illustrates a schematic representation of how a cornea was treated at only one half of the surface, leaving the other half untreated. This schematic representation maps to the pressure displacement curves shown in FIGS. 20B-H and 20J-M, which illustrate results of the inflation test performed on a half-treated cornea. Each displacement map lists the test pressure in units of kPa. It can be readily seen from FIGS. 20B-H and 20J-M that there is a marked change in the mechanical properties of the half of the cornea that has been treated as compared to the half that has not been treated.

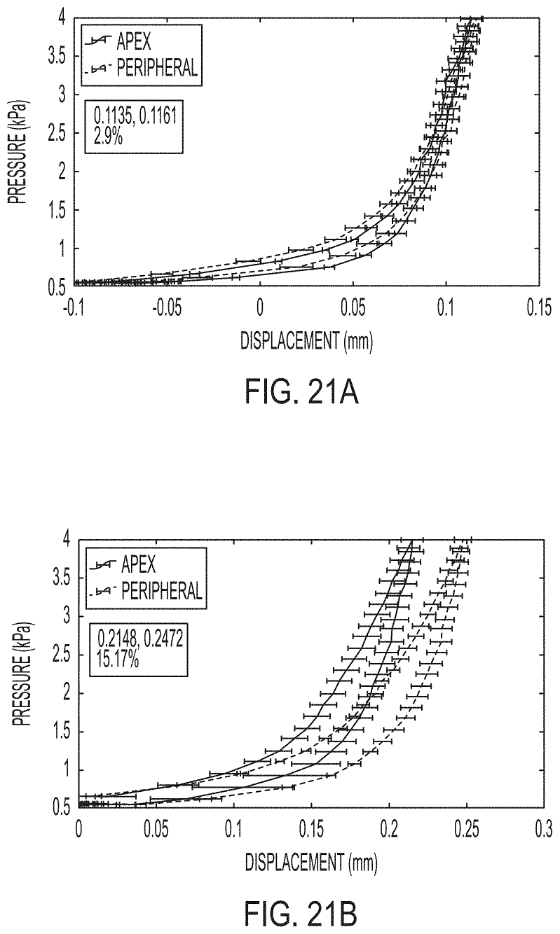

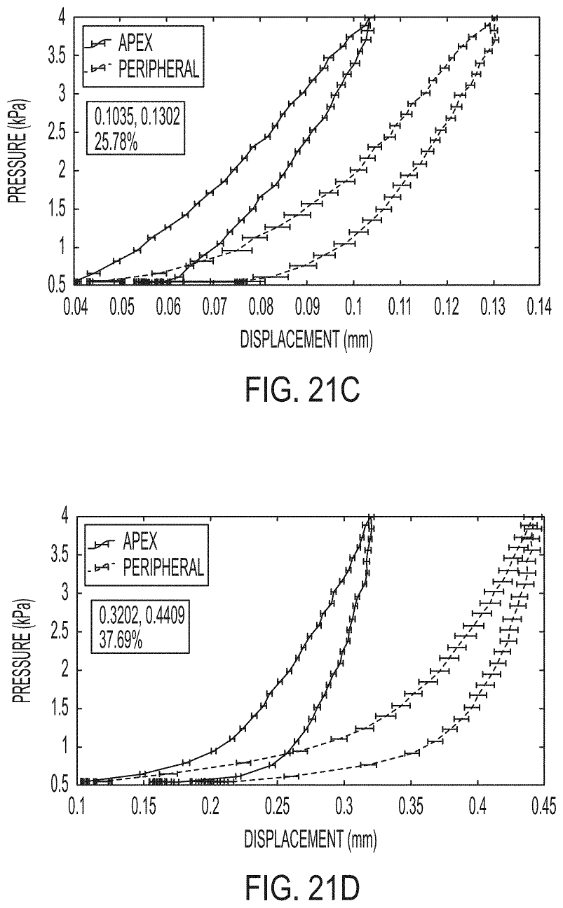

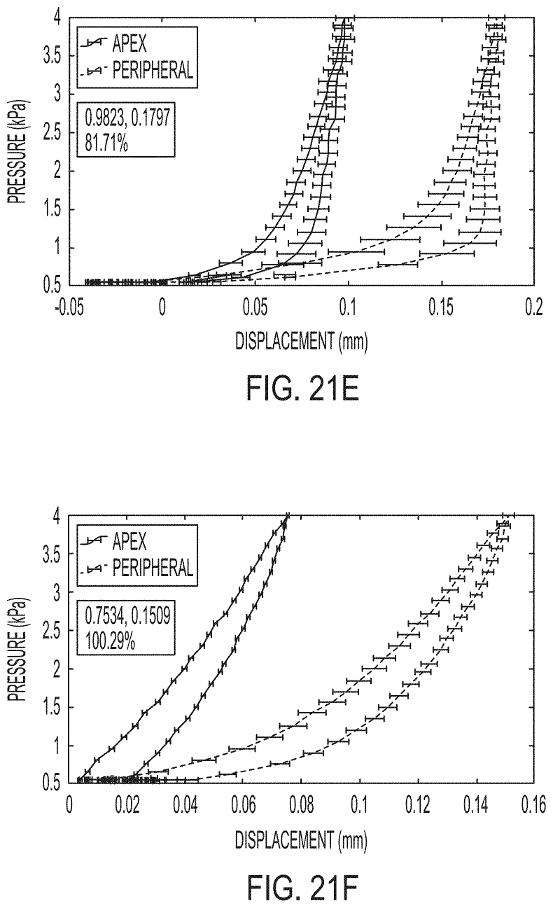

FIGS. 21A-F illustrate representative pressure-displacement curves subject to a loading-unloading rate of 0.00734 kPa/s at various points on six inflated corneas. Average displacements of 10 points from each treated apex and untreated peripheral region are graphed with error bars showing standard deviation. At maximum pressure point 4 kPa, percentage differences between the treated and untreated region are calculated and shown in the figures as follows: FIG. 21A represents the control, 2.29%; FIG. 21B represents 1-layer treated, 15.17%; FIG. 21C represents 2-layers treated, 25.78%; FIG. 21D represents 3-layers treated, 37.89%; FIG. 21E represents 4-layers treated, 81.71%; and FIG. 21F represents 5-layers treated, 100.29%.

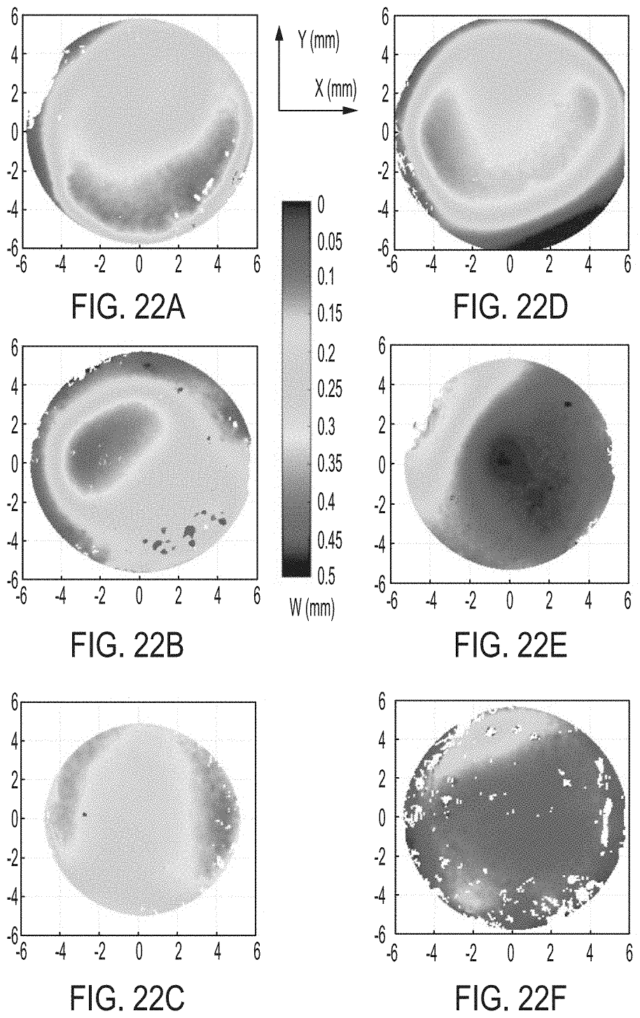

FIGS. 22A-F illustrate representative displacement position map of 6 inflated corneas at 4 kPa for: (FIG. 22A) control; (FIG. 22B) 1-layer treated; (FIG. 22C) 2-layers treated; (FIG. 22D) 3-layers treated; (FIG. 22E) 4-layers treated; and (FIG. 22F) 5-layers treated. In the figures, horizontal and vertical axes represent X and Y directions in millimeters, and the color bar represents changes in Z direction, W, in millimeters. Because individual corneas react to pressure differently due to inter-sample variances, there no visible trend in displacement for these 6 corneas was observed.

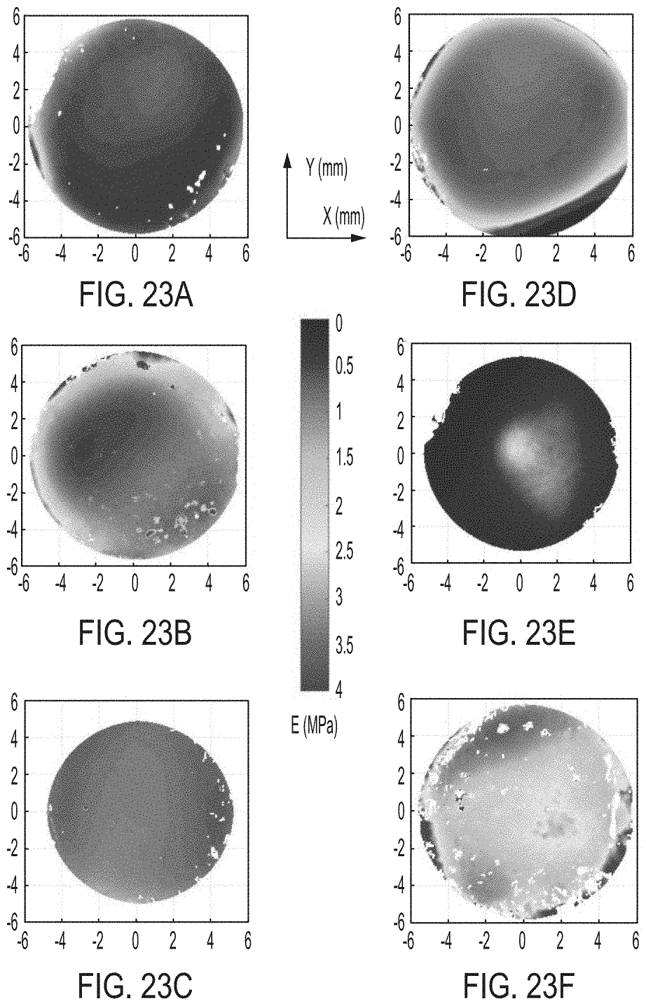

FIGS. 23A-F illustrate Youngs' Modulus maps of 6 inflated corneas at 4 kPa for different levels of treatment: (FIG. 23A) control; (FIG. 23B) 1-layer treated; (FIG. 23C) 2-layers treated; (FIG. 23D) 3-layers treated; (FIG. 23E)4-layers treated; and (FIG. 23F) 5-layers treated. Horizontal and vertical axis represents X and Y direction in millimeters, and the color bar represents Youngs Modulus, E, in MPa calculated using W and corneal thickness.

### Equivalents

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A method of altering curvature of a cornea, the method comprising:
receiving one or more measurements of topography of the cornea;
calculating a pattern defining locations and amounts of cross-linking required to achieve a desired level of vision correction, wherein the amounts of cross-linking are, at least in part, a function of a number of overlapping treatment layers having different z depths at a given coordinate; and
controlling a light source to apply light energy pulses to the cornea to cross-link collagen in accordance with the pattern.

2. The method of claim 1, further comprising:
receiving one or more measurements of thickness of the cornea,
wherein the amounts of cross-linking are a function of the thickness of the cornea.

3. The method of claim 1, wherein the light energy pulses are applied in the absence of an exogenous photosensitizer.

4. The method of claim 1, wherein the light energy pulses ionize water molecules within the cornea to generate reactive oxygen species.

5. The method of claim 1, wherein the light energy pulses have a wavelength that is not absorbed by amino acids in collagen.

6. The method of claim 1, wherein the light energy pulses have a wavelength that is absorbed by amino acids in collagen.

7. The method of claim 1, further comprising:
applying an exogenous photosensitizer to the cornea before controlling the light source.

8. The method of claim 7, wherein the exogenous photosensitizer is riboflavin.

9. The method of claim 1, wherein the light source is a laser.

10. The method of claim 9, wherein the laser is a femtosecond laser.

11. The method of claim 1, wherein the light energy pulses have an average power output between 10 mW and 100 mW.

12. The method of claim 1, wherein the light energy pulses have a pulse energy between 0.1 nJ and 10 nJ.

13. The method of claim 1, wherein the light energy pulses have a wavelength between 600 nm and 1600 nm.

14. A system for treating a cornea, the system comprising:
a light source configured to project light energy pulses onto at least a portion of the cornea; and
a controller programmed to receive one or more measurements of topography of the cornea;
calculate a pattern defining locations and amounts of cross-linking required to achieve a desired level of vision correction, wherein the amounts of cross-linking are, at least in part, a function of a number of overlapping treatment layers having different z depths at a given coordinate; and
control the light source to apply light energy pulses to the cornea to cross-link collagen in accordance with the pattern.

15. The system according to claim 14, further comprising:
laser modification optics adapted and configured to adjust laser output of the light source.

16. A method of treating a cornea of an eye, the method comprising:
flattening the cornea with a material that transmits light;
generating pulses with a tunable femtosecond laser system;
focusing the generated pulses on a focal volume at a specific depth within the cornea as measured from a surface of the eye;
moving the focal volume at the specific depth to define a treatment pattern; and
repeating the focusing and moving steps at multiple different depths.

17. The method according to claim 16, wherein the focusing is achieved by using an aspheric lens.

18. The method according to claim 16, wherein
the moving of the focal volume takes place at 30 mm/s in a direction parallel with the material used to flatten the cornea.

19. The method according to claim 16, wherein
adjacent ones of the multiple different depths are separated by 50 μm.

20. The method according to claim 16, wherein
the flattening the cornea includes pressing a glass coverslip against the cornea,
the generating pulses is performed with a temporal pulse width of 140 fs at 80 MHz repetition rate with central wavelength set to 1060 nm, and
the treatment pattern is a zig-zag pattern.

21-22. (canceled)
